(19) United States
(12) Patent Application Publication
(10) Pub. No.: US 2003/0124643 A1
(43) Pub. Date: Jul. 3, 2003

(54) Method and kit forrapid concurrent identification and antimicrobial susceptibility testing of microorganisms from broth culture

(76) Inventor: Read Robert Taintor, North Salt Lake, UT (US)

Correspondence Address: Read Robert Taintor, 98 Mason Lane, North Salt Lake, UT, 84054, US

(21) Appl. No.: 09/998,638

(22) Filed: Dec. 3, 2001

(51) Int. Cl. C12Q 1/04; C12Q 1/08

(52) U.S. Cl. 435/40; 435/34

(57) ABSTRACT

A Method and Kit for performing concurrent identification testing and antimicrobial susceptibility testing from broth culture (90) are described. Broth (82) incubation is generally 4 to 6 hrs providing adequate numbers of microorganisms for inoculating a multi-chambered kit plate (80) comprising enriched, differential, selective, differential-selective, single-purpose and susceptibility media. Several dilutions are prepared from the cultured broth, for inoculation of the kit plate (80). The more dilute concentration (140) produces individual colonies of microorganisms, for identification testing. This isolation makes an initial isolation step unnecessary. The heavier concentration dilution (96) provides inoculation for antimicrobial susceptibility tests and other identification tests. In addition, antimicrobial susceptibilities are shown valid even when several different microorganisms coexist in the same test chamber. The method is fast for bacteria, providing identification and susceptibility data in 24 hrs. The kit is complete, except for an incubator and microscope. The method is simple to perform and can be utilized almost anywhere.

### BACKGROUND

1. Field of Invention

This invention relates to the concurrent identification (ID) and antimicrobial susceptibility testing (AST) of an unknown microorganism or microorganisms manually determined using one kit from specimen to results in normally 24 hrs.

2. Prior Art

The diagnosis of infectious diseases has traditionally relied upon microbiological culture methods to identify the organism responsible for the infection and then determine the appropriate antimicrobial treatment. These methods continue to be important for analysis, despite recent advances in molecular and immunological diagnostics. While the development of rapid and automated methods has served to increase the efficiency of microbiological analysis, traditional quantitative culture methods remain critical for definitive diagnosis of infections. (Baron & Finegold, Diagnostic Microbiology, 8^{th} ed. C. V. Mosby, [1990], p 253. These traditional methods are even more valuable in countries unable to afford newer methods including automated methods. In addition, many areas of the world are devoid of any adequate clinical microbiology facility.

Throughout history, humanity has fallen victim to pandemics of cholera, plague, influenza, typhoid, tuberculosis and other infectious maladies so widespread, that few people made it into middle age. As recently as the 19^{th} century, the average life span in Europe and North America was around 50 years. It was a world in which the likelihood of dying prematurely from infectious diseases was as high as 40%, and where women routinely succumbed during childbirth to infections easily curable by today's standards. In underdeveloped nations, the situation was even worse, with one caveat: unlike industrialized nations, conditions in underdeveloped nations never really improved. In poorer nations today, infectious diseases, both major and seemingly minor, further contribute to premature death and the ongoing misery of underprivileged populations.

The emergence of multi-resistant bacteria (antibiotic-resistant bacteria) is also a worldwide concern. Antibiotics are indiscriminately used, and this has contributed to the rise of antibiotic resistance in a variety of bacteria, including species of Enterococcus, Staphylococcus, Pseudomonas, and the Enterobacteriaceae family. The emergence of antibiotic-resistant organisms is a result of the over-use of broad-spectrum antibiotics. There is also concern that inappropriate veterinary use of antibiotics may lead to development of antibiotic resistant bacteria, which could in turn infect humans.

Traditional Specimen Collection and Transport

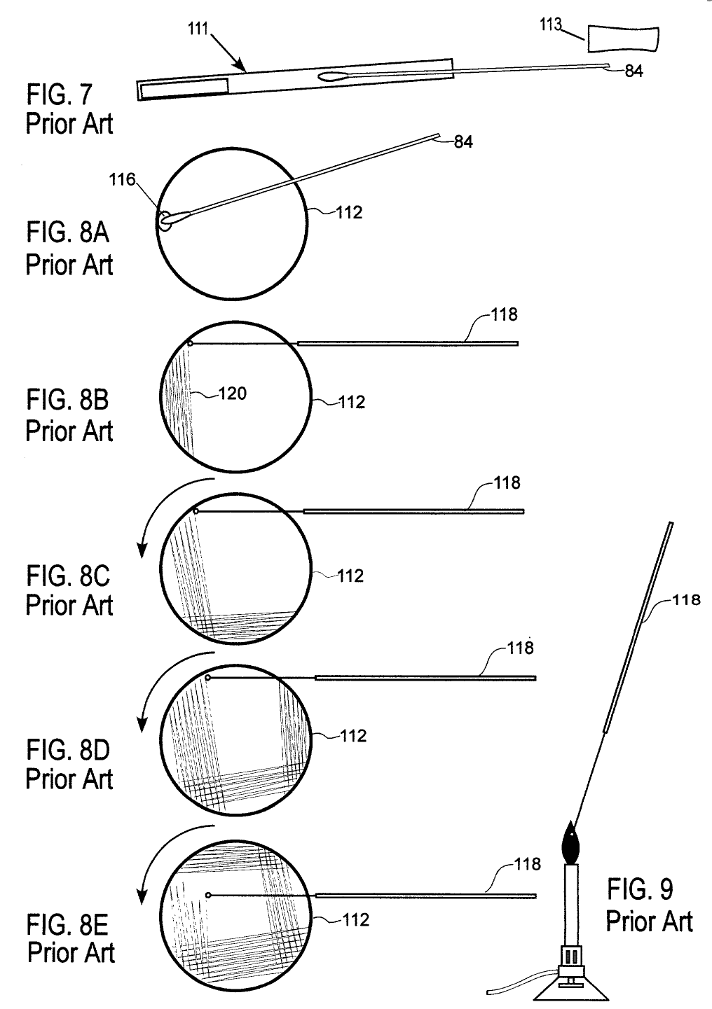

The specimen must be material from the actual infection site. Once collected, it is necessary to maintain the sample as near to its original state as possible with minimum deterioration. The transport system consists of a protective container, transport medium and the culture swab (FIG. 7). A problem with the use of a holding or transport medium is that it may jeopardize the recovery of certain strains. A major task is to reduce the time delay between collection of specimens and inoculation onto microbiological culture media. The transport container is constructed to minimize hazards to specimen handlers. It is best to minimize adverse environmental conditions, such as rapid changes in pressure, exposure to extremes of heat and cold or excessive drying. The transport of fluid specimens to the laboratory must be done as quickly as possible. It is recommended that a 2-hr maximum time limit be imposed between collection and delivery of specimens to the laboratory. This limit poses a problem for specimens collected any distance from a clinical microbiology laboratory.

Microbiological Culture Media and their Usefulness

A satisfactory microbiological culture medium must contain available sources of water, vitamins, inorganic phosphate and sulfur, trace metals, carbon and nitrogen. These needs are supplied from a number of sources. In addition, there are agents that manipulate what organisms can grow and others that enhance identification. The following is a list of common media constituents with their sources in parenthesis: (1) Amino-nitrogen (peptone, protein hydrolysate, infusions and extracts), (2) Growth factors (blood, serum, yeast extract or vitamins, NAD), (3) Energy sources (sugar, alcohols, and carbohydrates), (4) Buffer salts (Phosphates, acetates and citrates), (5) Mineral salts and metals (phosphate, sulfate, magnesium, calcium, iron), (6) Selective agents (chemicals, antimicrobials and dyes), (7) Indicator dyes (phenol red, neutral red), and (8) Solidifying agents (agar, gelatin, alginate, silica gel, etc.). The media can be in a liquid or a solid form. Solid media provides for the isolation of microorganisms contained in a mixture of different microorganisms. Liquid media, referred to as “Broth”, can provide a nutritionally rich environment which is more accessible to the individual cells than solid media. This allows the microorganisms to grow rapidly but they are not isolated from each other. Brain Heart Infusion Broth is a rich media supplying many of the compounds that the cell would otherwise have to synthesize. This allows the cell to devote more of its energy to growth, which is another reason for their faster growth in liquid media.

A selection of the appropriate solid culture media for microbiological test(s) is made according to the particular specimen type. Several hundred culture media are commercially available. Various culture media have been developed to serve specific purposes such as Mueller Hinton agar, as an antimicrobial susceptibility testing media. The media comprising identification testing media can be divided into five groups: ENRICHED MEDIA have special additives to support pathogens having fastidious growth needs. Examples of media include sheep blood agar and Brain Heart Infusion Broth. DIFFERENTIAL MEDIA allows differentiating of groups of microorganisms based on indicator color changes (such as pH) in the culture medium that take place as a result of biochemical reactions associated with microorganism growth. Separating organisms that ferment the sugar lactose, for example, from those that do not, is one example of differential media. SELECTIVE MEDIA support the growth of certain microorganisms of interest, while suppressing the growth of others. Azide blood agar is an example. Gram-positive organisms grow on this media whereas gram-negative organisms do not. DIFFERENTIAL-SELECTIVE MEDIA combine the last two characteristics, to allow the selective growth and rapid differentiation of major groups of bacteria. These media are widely used for gram-negative bacilli (rods). MacConkey and Hektoen are examples. SINGLE PURPOSE MEDIA isolate one specific type of microorganism. Bile esculin azide agar is an example of this media. Enterococcus and group D streptococcus grow and cause the formation of a dark brown or black complex in the agar. In the microbiology laboratory, every attempt is made to use well-trained personnel, under close supervision, for the processing of specimens. Errors or misjudgments made during this link in the diagnostic process such as improper choice of media can negate all the expertise one may apply in the reading and interpretation of cultures. Expert microbiologists are caught short in making a definitive diagnosis because inadequate or incorrect media was selected for a specimen.

Techniques for Culturing Specimens and Ultimate Identification of the Microorganism(s)

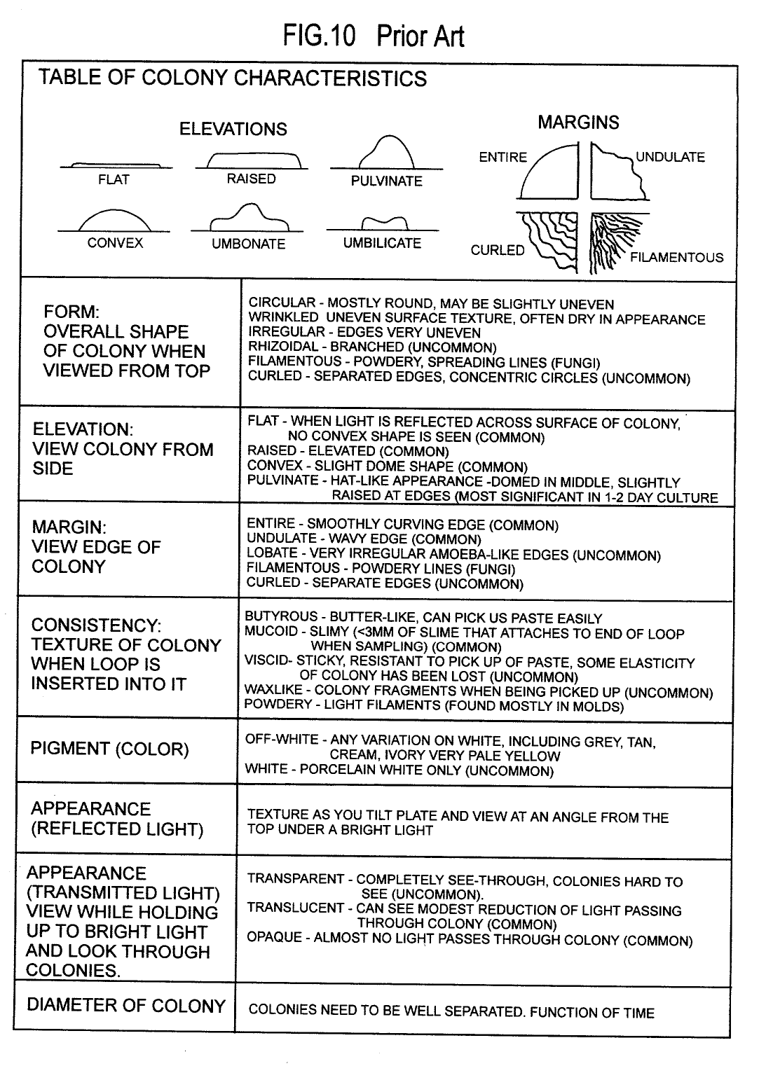

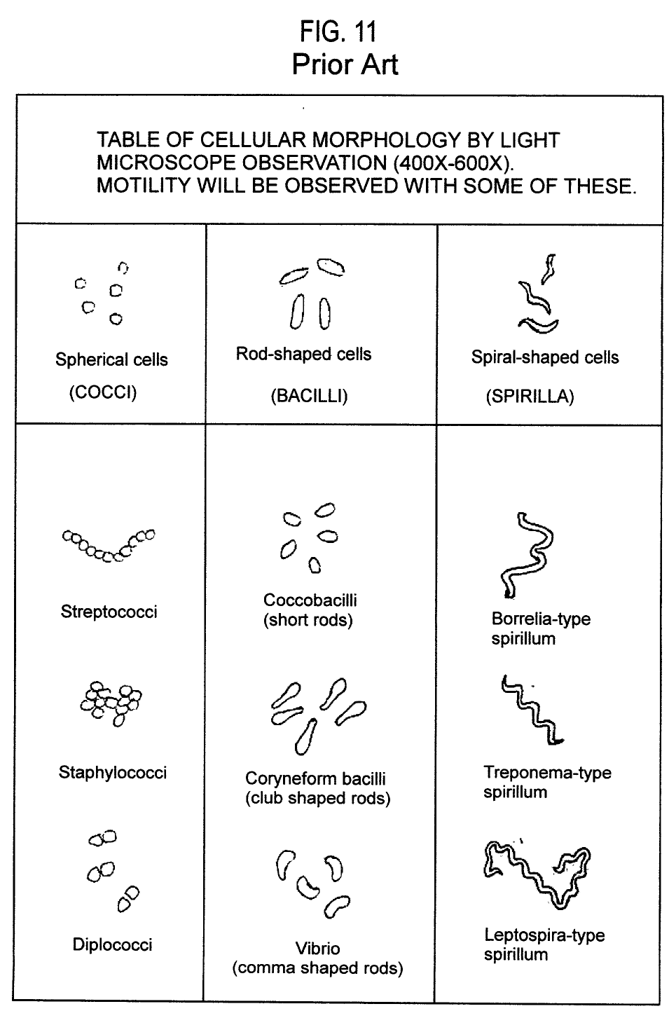

The equipment required for the primary inoculation of specimens consists of several microbiological agar-based media plates and a nichrome or platinum inoculating wire or loop (see FIGS. 8B-8E). The plates generally have a shelf life of from one to two months. Streaking out the specimen spreads the microorganisms across the surface of the culture medium. This results in isolated colonies. The first step is to touch and roll the tip of the swab 84 containing the specimen 116 on the surface of the medium (FIG. 8A). Then, using an inoculating loop 118 that has been flamed to sterilize it (FIG. 9), streak the primary inoculum 116 by spreading it out in the first quadrant (FIG. 8B). Re-sterilize the loop 118 and cool. Streak the inoculum from the first quadrant into the second quadrant (FIG. 8C). Repeat the process for the other two quadrants (FIGS. 8D-8E). Incubate the plate following the placement of a lid for 18 to 24 hrs. The preceding method is the standard prior art method for isolating microorganisms where at each new streaking they become further diluted until they finally become isolated from one another. As the isolated microorganisms grow on the solid medium, they form a mass called a colony. This mass of cells originated from a single cell and now may consist of hundreds of thousands of cells. These colonies have distinct characteristics that are a clue in the process of identifying the microorganism (see FIG. 10). The sub-culturing of the isolated colonies to additional media produces pure cultures. The microscopic examination of a suspension of bacteria from a colony reveals (a) cellular morphology, (b) cellular arrangement, and (c) motility. These features (See FIG. 11) add additional pieces to the ID puzzle. A gram stain of the sample may also assist the analyst in getting closer to a characterization of the organism. The gram stain is not foolproof however, and can be occasionally misleading because the staining is frequently dependent upon the age of the colony.

The testing of certain enzyme systems unique to each species provides further clues to the ID of an unknown. Another basis for ID is the culture requirements, which includes the atmospheric needs of the organism as well as nutritional requirements and ability to grow on different kinds of media. A further basis of ID in regards to the biochemical characteristics includes the mode of carbohydrate utilization, catalase reactions of gram-positive bacteria and oxidase reactions of gram-negative bacteria. ID to the species level is based on a set of physiological and biochemical characteristics including the degradation of carbohydrates, amino acids, and a variety of other substrates.

Commercial kits perform a number of various biochemical reactions. The results of these reactions can reveal unique patterns for ID. Some systems are automated and others are manual. A problem with manual systems is the limited scope in terms of the organisms they target for ID. Additionally it is necessary to first isolate the organism of interest from other microorganisms in an 18 to 24 hr isolation step as described above ([0008]) before applying the organism to the manual or automated ID system. For example, the manufacturer bioMerieux Vitek® markets the following manual systems (listing the target organisms): API 20C AUX (yeasts), API 20E (Enterobacteriaceae and non-fermenting gram-negative bacteria), API 30 Strep. (Streptococcus and Enterococcus), API Coryne (Corynebacteria and coryne-like-organisms), API 20 NE (Gram-negative non-Enterobacteriaceae), API Rapid 20E (Enterobacteriaceae), and API Staph (Staphylococcus and micrococcus). Judgment must be made by the microbiologist as to which isolate to test and the proper ID system to use. This is another source of possible error.

Antibiotic Susceptibility Testing Using the Disk Diffusion Susceptibility Test

The prior art calls for initial isolation and identification of the organism first and then, if deemed appropriate, i.e. where a pathogen is identified, performing an antimicrobial susceptibility test. In addition, the analyst must decide which microorganism is responsible for the clinical disease in mixed cultures. There are a number of different ways of doing antimicrobial susceptibility testing (AST). Two of them are disk-diffusion and micro dilution.

In recent years, there has been a trend toward the use of commercial broth micro dilution and automated instrument methods instead of the disk-diffusion procedure. However, there may be renewed interest in the disk-diffusion test because of its inherent flexibility in drug selection and low cost. The availability of numerous antimicrobial agents and the diversity in antibiotic formularies in different institutions have made it difficult for manufactures of commercial test systems to provide standard test panels that fit every facility's needs. Thus, the inherent flexibility of drug selection provided by the disk-diffusion test is an undeniable asset of the method. It is also one of the most established and best proven of all AST tests and continues to be updated and refined through frequent National Committee for Clinical Laboratory Standards (NCCLS) publications. Furthermore, clinicians readily understand the qualitative interpretive category results of susceptible, intermediate, and resistant provided by the disk test. It is an ideal method when doing manual diagnostic microbiology

Procedure for Disk-Diffusion Test

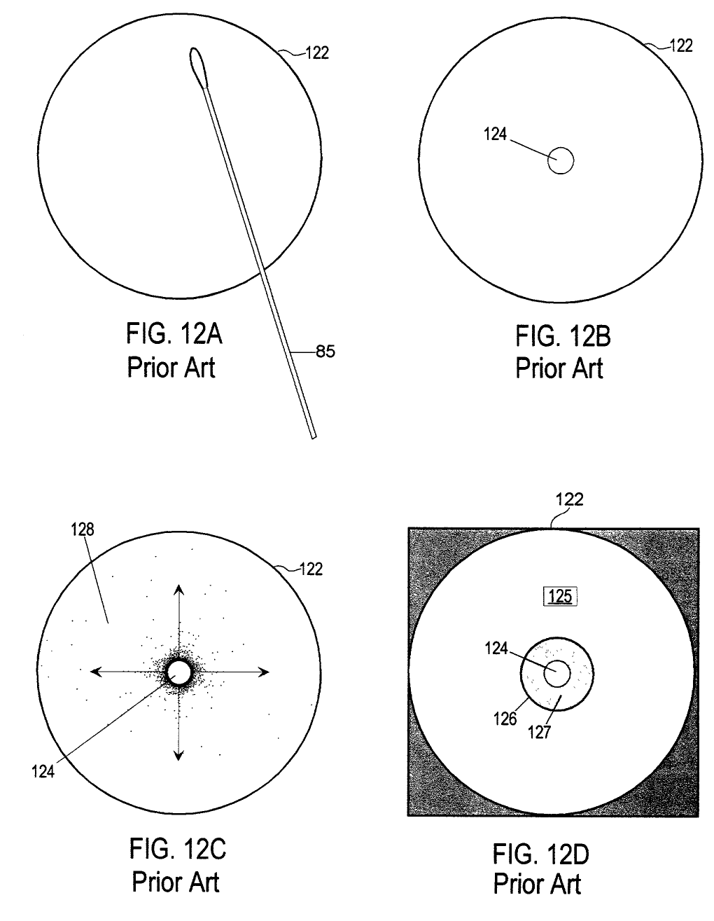

The initial isolation step results in colonies formed from a single microorganism. The analyst then transfers like colonies into growth broth. The broth is incubated at 35° C. for 2 to 8 hr until growth reaches the turbidity at or above that of a McFarland 0.5 standard 94. This turbidity is equivalent to 1.5×10⁸ colony forming units (CFU)/ml. McFarland standards are prepared using different amounts of barium sulfate in water. This salt is insoluble in water and forms a very fine suspension when shook. Within 15 minutes of adjusting turbidity, a cotton swab 85 transfers this inoculum to a Standard Susceptibility Dish 122. The entire surface of the Mueller-Hinton plate is swabbed three times, rotating the plate approximately 60 degrees between streaking to ensure even distribution (FIG. 12A). The plate stands for 3 to 15 minutes before AST disk 124 is applied. Apply to the agar surface with a dispenser or manually with sterile forceps. Apply gentle pressure to ensure complete contact of the disk with the agar. (FIG. 12B showing one disk added). Incubate for 16 to 18 hours at 35° C. in an ambient-air incubator.

FIG. 12C illustrates the basic principle of the disk-diffusion method of AST. As soon as the antibiotic-impregnated AST disk 124 is exposed to the moist agar surface, water is absorbed into the filter paper and the antibiotic 128 diffuses into the surrounding medium. The rate of extraction of the antibiotic out of the disk is greater than its outward diffusion into the medium, so that the concentration immediately adjacent to the disk may exceed that in the disk itself. As the distance from the disk increases, however, there is a logarithmic reduction in the antibiotic concentration. If the plate has been previously inoculated with a bacterial suspension, simultaneous growth of bacteria occurs on the surface of the agar. When a critical cell mass of bacteria is reached, the inhibitory activity of the antibiotic is overcome and microbial growth occurs. The time (critical time) required to reach the critical cell mass (4 to 10 hours for commonly tested bacteria) is characteristic of each species but is influenced by the composition of the medium and temperature of incubation. The depth of the agar will affect the lateral extent of antimicrobial diffusion before the critical time is reached because diffusion occurs in three dimensions. The points at which the critical cell mass is reached appears as a sharply marginated circle (margin 126), of microorganism growth 125, with the middle of the disk forming the center of the circle if the test has been performed properly (see FIG. 12D). The concentration of diffused antibiotic at this margin 126 of growing and non-growing bacteria 127 is known as the critical concentration. This concentration approximates the minimal inhibitory concentration (MIC) obtained in dilution tests. The Minimal inhibitory concentration (MIC) is the lowest concentration of a chemotherapeutic agent that will prevent growth of the test microorganisms. The disk-diffusion test that has become standard in the United States is based on the work of Bauer, Kirby and coworkers. The zone size observed in a disk-diffusion test has no meaning in and of itself. The interpretative standards provided by the NCCLS show the correlation between zone sizes and MICs of those species tested by disk-diffusion method.

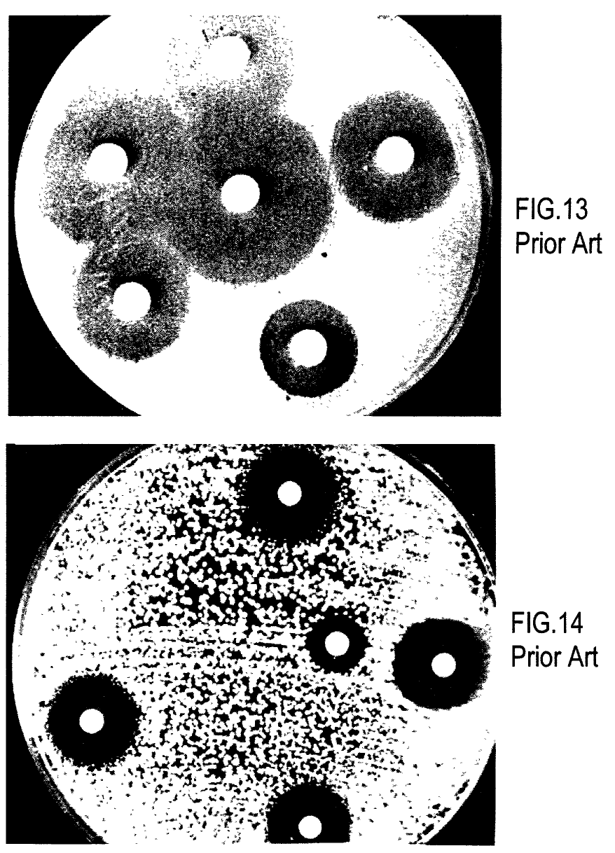

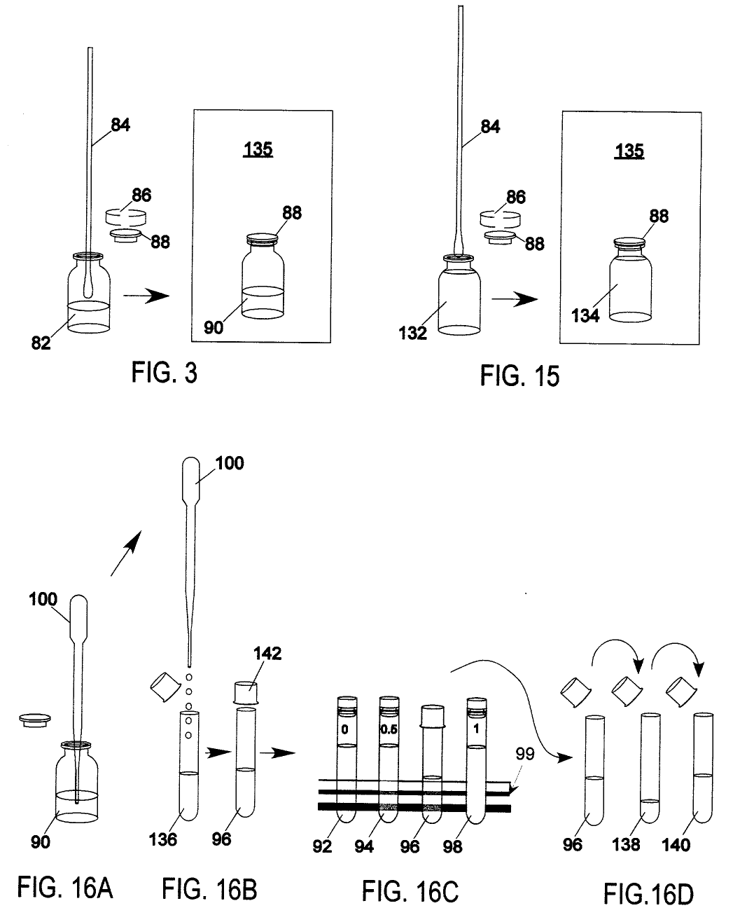

FIG. 13 shows a poorly prepared AST plate with objectionable overlapping of the zones of growth inhibition from adjacent disks. FIG. 14 shows a poorly streaked AST plate with uneven growth. The zone margins are indistinct, compromising accurate measurement.

A patent search was performed to determine if there was a patented method whereby ID and AST determinations could be done concurrently on a mix of non-isolated organisms (such as a broth culture) from the same specimen or sample either manually or automated. None matched the forgoing criteria.

A distinct disadvantage of the above prior art is the total time that it takes from obtaining the culture through performing ID and AST. At least three days transpire before results are available. Another disadvantage is the expense to process the specimen using prior art. A further disadvantage of the prior art is the number of steps involved in performing the tests, which increases the likelihood of human error. In addition, the agar-based microbiology media that is used in the testing has a limited shelf life of one to two months at most.

Diagnostic microbiology prior art is an involved process that requires a substantial investment in terms of time, resources and expertise. There does not currently exist in the prior art, a method or kit that can accomplish both a rapid, straightforward ID and AST of an unknown microorganism or microorganisms from a single sample, where a prior isolation step is not required first.

### SUMMARY

The present method and kit relates to the ID of microorganisms and concurrent or consecutive determination of antimicrobial susceptibilities (AST). The process is novel and unconventional because the testing is done directly from an initial broth culture with no isolation step needed. The method and kit offers quick characterization of microorganisms, in one-third the time of standard manual methods.

The Kit employs a disposable multi-chambered plate (kit plate) with enriched, differential, selective, and differential-selective media in addition to AST medium. Broth medium is provided for growing up the microorganisms for eventual dilutions and inoculation onto the kit plate. AST disk-quarters are included as well as several biochemical reagents for additional testing. The shelf life of the kit is at least 5 months from date of manufacture when stored at 4° C.

### OBJECTS AND ADVANTAGES

Accordingly, several objects and advantages of the present invention are:

(a) To provide a method and kit for an in-house or in-the-field characterization of unknown microorganisms. The Kit comes complete to perform the testing, except for an incubator and a simple microscope. A portable incubator can be operated from any direct current source such as an automobile battery. “Microorganism” is understood to mean, in particular, microbes, bacteria and yeasts. The kit is well suited in areas where microbiology laboratories are scarce or unavailable. In addition, the kit serves to obtain rapid AST information.

Microorganisms such as Anthrax (Bacillus anthracis) can be determined concurrently with drug susceptibility testing within 24 hrs.

(b) To improve the situation throughout our world in regards to rising resistance to antibiotics. Many antibiotics are no longer effective against certain strains of bacteria. In fact, AST is useful and important for the common microorganism species that are not predictably susceptible to drugs of choice because of acquired resistance mechanisms (e.g., members of the Enterobacteriaceae, the Pseudomonas species, Staphylococcus species, Enterococcus species, Streptococcus pneumoniae, Haemophilus influenzae, and Neisseria gonorrhoeae). A recent editorial in the British Journal of Medicine states: “Research is also a cornerstone in the fight against bacterial resistance. We have to improve our understanding of microorganism flora, the evolution of resistance, and the mechanisms of transmissibility of resistant bacteria. New diagnostic technologies to enable rapid ID of viral and bacterial infections are also necessary: for too long it has been easier for clinicians to prescribe an antibiotic than to make a specific diagnosis”. The kit and method accomplishes the rapid ID of microorganism infections needed for making a specific diagnosis. In addition, results of antimicrobial susceptibilities complete the picture.

(c) To provide a method and kit where an ideal specimen from the site of infection or a microorganism-containing sample can be immediately applied to broth culture media. The use of transport media is unnecessary. Therefore, the specimen is not subjected to time delays; possible adverse environmental conditions or excessive drying that would compromise its integrity. Fluid specimens can also be immediately processed. In addition, a more rapid result is realized with this system due to immediate inoculation of the specimen to broth culture. The microorganism sample is ready for dilution and inoculation onto the multi-chambered kit plate within 4 to 8 hrs following broth culture incubation.

(d) To provide a method and kit comprising a multi-chambered, easily visualized culture kit plate comprising a battery of different media with diagnostic functionality. The miniaturization of the media is very cost effective. The multi-kit plate media performs the ID and AST of gram-negative and gram-positive organisms. One chamber is devoted to fungi determination. Any errors or misjudgments in the prior art of media selection do not exist with the present system. An appropriate selection of medium is already incorporated in the design of the multi-chambered kit plate. This insures that the user of the kit will not be caught short in making a definitive diagnosis due to incorrect media selection.

(e) A special dilution method utilized with the kit simplifies the inoculation of the microorganism sample to the various media of the kit plate. Time is of the essence with an infection. The prior art of streaking each diagnostic plate for isolation of the organisms becomes unnecessary. The present kit utilizes a liquid dilution to a standard equivalent from a starting broth culture. A further dilution added to the kit results in individual colonies in the various media test chambers. A magnifying lens such as a microscope 10×objective turned backwards provides a good view of the morphology and chemistry of the microorganism's colony on the various media. This makes the prior art isolation step unnecessary. Isolation and ID takes place together in the same chamber at the same time. The method is very reproducible and the faster growing organisms such as members of the Enterobacteriaceae family can produce discernable colonies within 12 to 18 hrs.

(f) To provide a method and kit for determination of unlike microorganisms, such as gram-negative as well as gram-positive, at the same time. Thus, it becomes unnecessary to use different manual ID systems as described in the prior art ([0010]). This saves money as well as time. Another advantage is that errors in judgment are avoided with regards to the selection of the appropriate kit or kits, for analysis. This prior art selection process would follow an initial isolation step (streaking, incubating and assessing) on a microbiological plate.

(g) To provide a method and kit for concurrent ID and AST. The Kirby-Bauer disk-diffusion method used with this kit allows for flexibility in terms of choice of antimicrobial agents. The method of placing the antibiotics into the AST test chambers is quick, using a novel method. The resultant zone size is measured as radius whereas in a prior art standard method, zone size is measured as diameter which is exactly twice the value of the miniature assay. This allows the use of the NCCLS interpretative standards charts divided by 2. A third advantage is that the test chambers containing the AST media are well covered with a uniform inoculum of bacteria and produce a lawn of microorganism growth unlike the larger dishes that are prone to unevenness. In addition, since each antimicrobial is in its own chamber, there is no overlapping of the zones of growth inhibition from adjacent disks. A forth-important advantage is manifest when there is more than one organism on the kit plate. When more than one zone is evident, morphology of the more resistant organism (inner zone) can to observed by taking a sample of inner zone bacteria and observing microscopically. It has been observed that routine cultures that grow three or more organism types should be discounted. Specimens obtained from non-sterile sites most commonly represent colonization or contamination.

(h) To provide a kit and method for ID and AST that can yield results in one-third the time of the prior art methods. This method and kit can accomplish both rapid, straightforward ID and AST of an unknown microorganism or microorganisms from a single sample, where a prior isolation step is not required. Therefore, the patient can start on the correct antibiotic by the next day and avoid having to take an incorrect empirical antibiotic for a 3-day period, as would be the case in the prior art. Where the infection is life threatening, it is a powerful approach to the problem.

(i) To provide a kit and method for ID and AST where the kit plate component has a shelf life of at least 5 months when stored at 4° C.

(j) Further objects and advantages of the method and kit will become apparent from a consideration of the drawings and ensuing description.

### DRAWINGS

### FIGURES

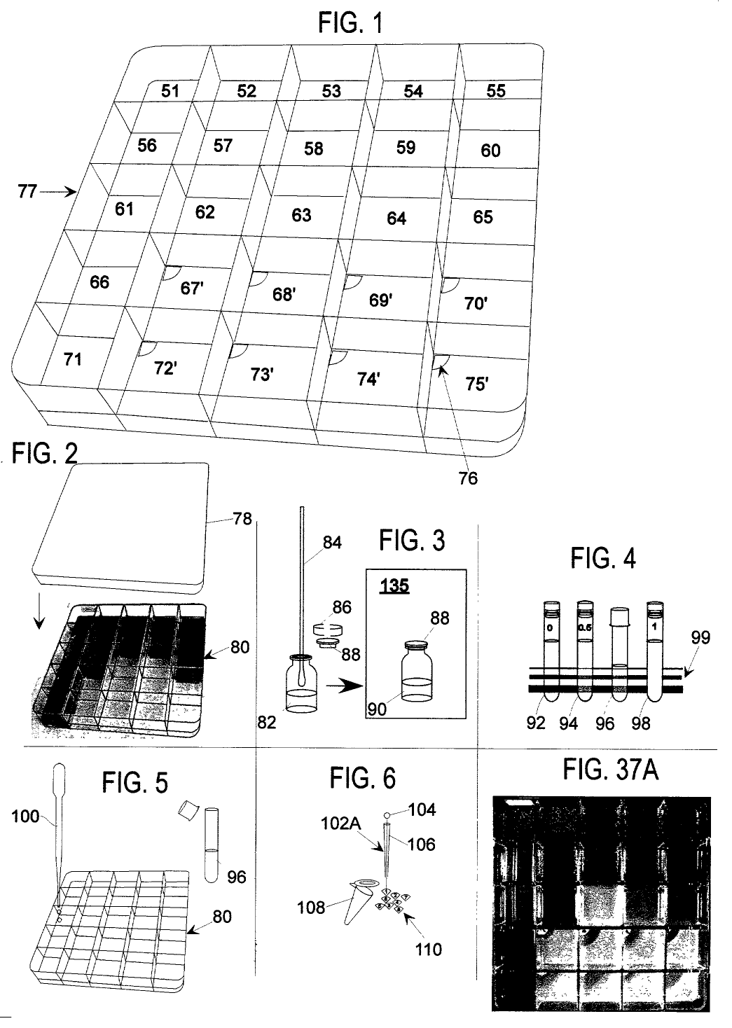

FIG. 1 shows a perspective view of the preferred embodiment of the present invention, a multi-chambered kit plate with square test chambers. Eight of the test chambers show antimicrobial disk-quarters in the corners of the test chambers.

FIG. 2 shows preferred embodiment kit plate and lid with various selective, differential and non-selective agar-based solid media. In addition, the lower right eight test chambers contain Mueller Hinton agar.

FIG. 3 shows the preferred embodiment of the method of inoculation of Brain Heart infusion broth from a specimen and subsequent incubation called the initial broth culture

FIG. 4 shows three McFarland turbidity standards with a dilution of the incubated broth.

FIG. 5 shows the preferred embodiment method of applying a 0.5 McFarland equivalent dilution of incubated microorganisms to the bottom three rows of the kit plate.

FIG. 6 shows the preferred embodiment device used to apply the antimicrobial disk-quarters to the susceptibility test chambers and a depiction of a set of antimicrobial disk-quarters.

FIG. 7 shows a typical culture transport system

FIGS. 8A to 8E shows the prior art addition of a microorganism sample to a standard solid media plate and subsequent streaking process which dilutes out the microorganism.

FIG. 9 shows a Bunsen burner

FIG. 10 shows a table of various microorganism colony characteristics from the prior art.

FIG. 11 shows a table of various microorganism cell morphologies as observed with light microscope from the prior art.

FIGS. 12A to 12D shows the method and principle of the prior art AST Kirby-Bauer disk-diffusion test.

FIG. 13 shows a poorly prepared Kirby-Bauer disk diffusion test with overlapping zones from prior art.

FIG. 14 shows a poorly prepared AST test with under applied bacteria from prior art.

FIG. 15 shows one embodiment of the inoculation of Thioglycolate broth and subsequent incubation.

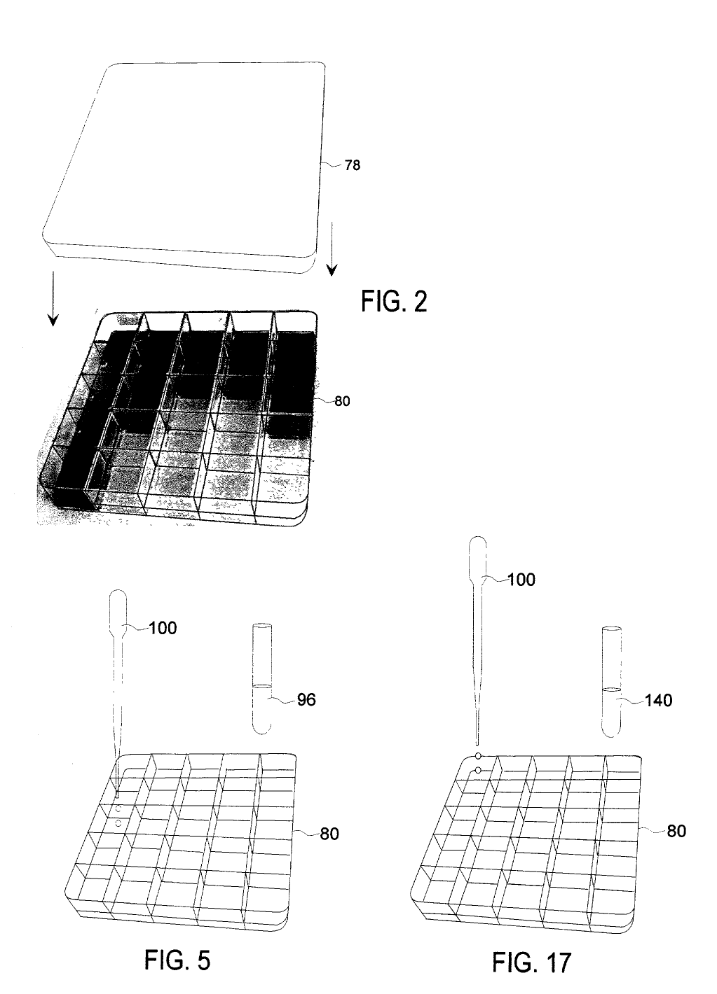

FIGS. 16A to 16D shows the preferred embodiment method of preparing dilutions directly from initial broth culture (without an isolation step) for inoculating the kit plate.

FIG. 17 shows the preferred embodiment method of applying a further microorganism suspension dilution (1 to 1000 of the 0.5 McFarland equivalent dilution) of to the top two rows of the kit plate.

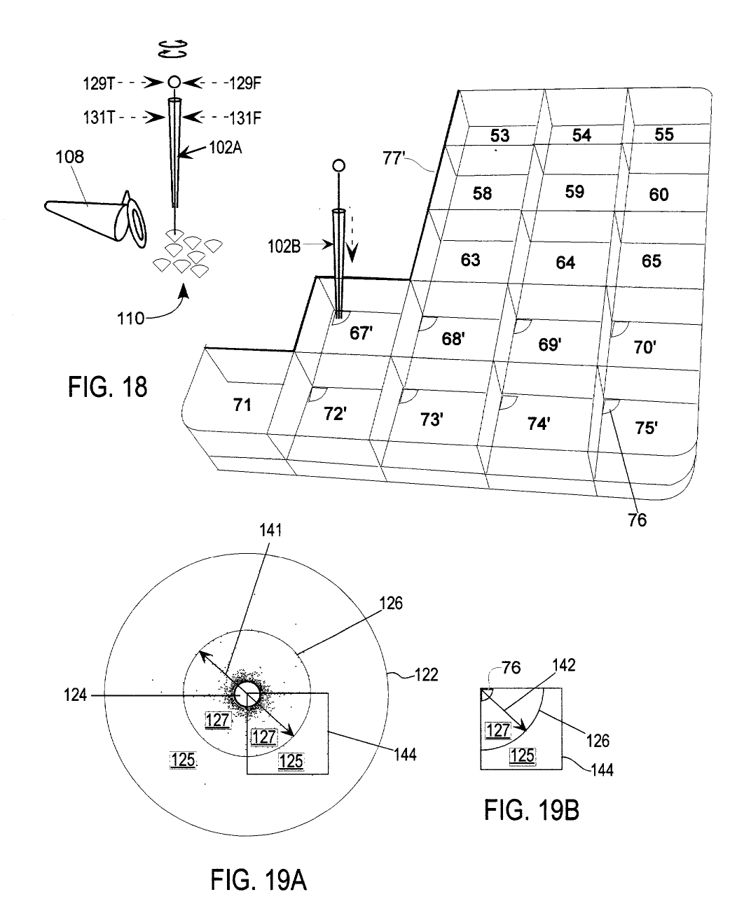

FIG. 18 shows the preferred embodiment method of applying antimicrobial disk-quarters to the corners of the Mueller Hinton test chambers using the placement device.

FIGS. 19A and 19B shows the principle of the standard Kirby-Bauer disk-diffusion test and illustrates how the preferred embodiment system measures exactly one-half of the measurement of the prior art standard method.

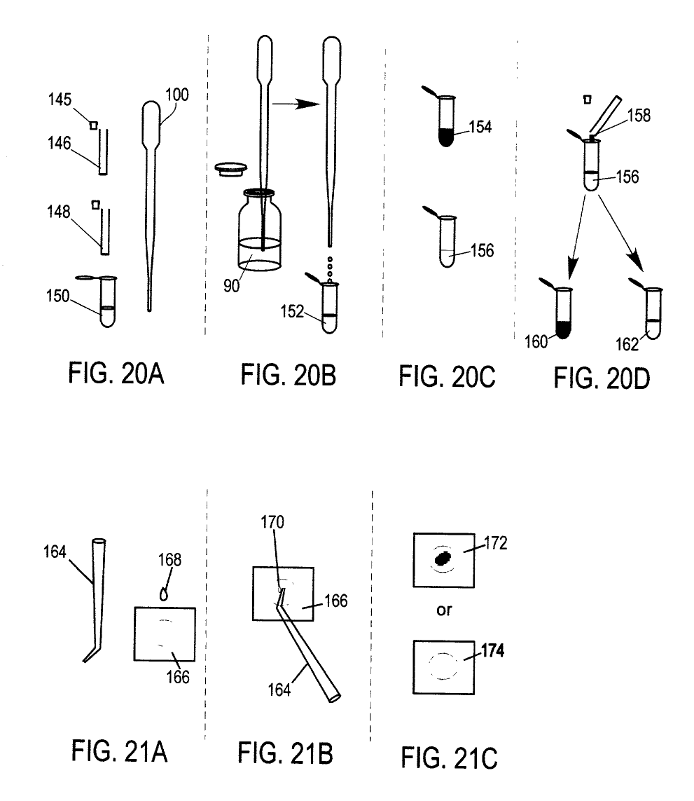

FIGS. 20A to 20D shows the preferred embodiment method for determining nitrate reductase activity as part of the kit.

FIGS. 21A to 21C shows the preferred embodiment method for determining cytochrome oxidase activity as part of the kit.

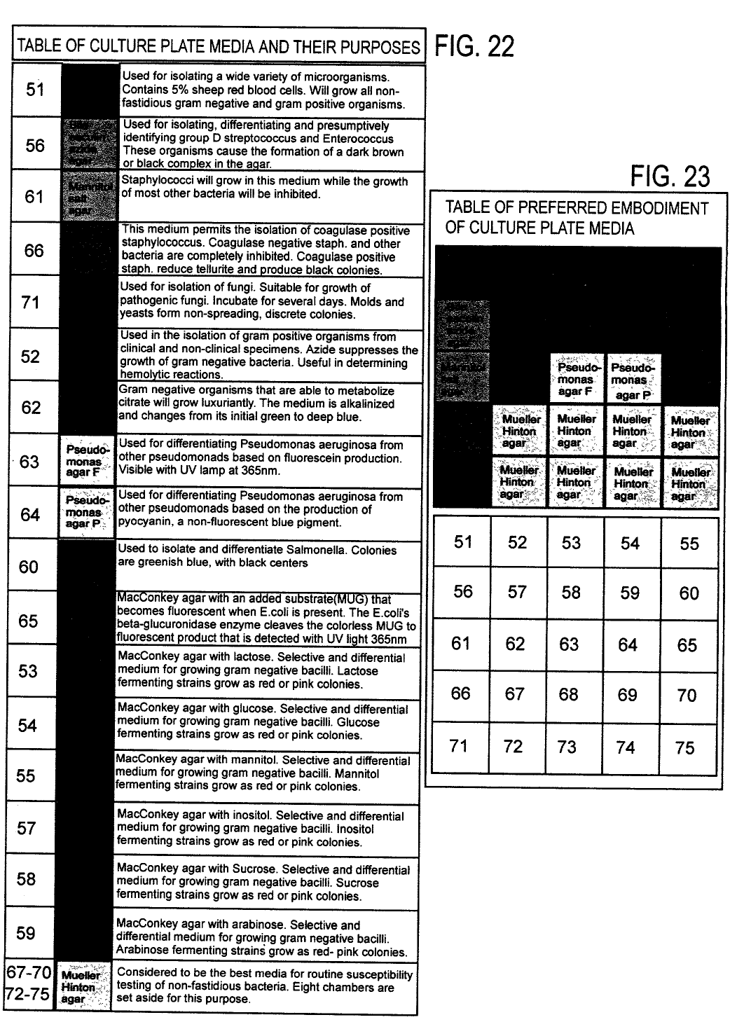

FIG. 22 shows a table of the preferred embodiment media components of the multi-chambered kit plate and associated reference numerals, where media formulations themselves are prior art.

FIG. 23 shows a table of the kit plate media layout of the preferred embodiment with the associated reference numerals.

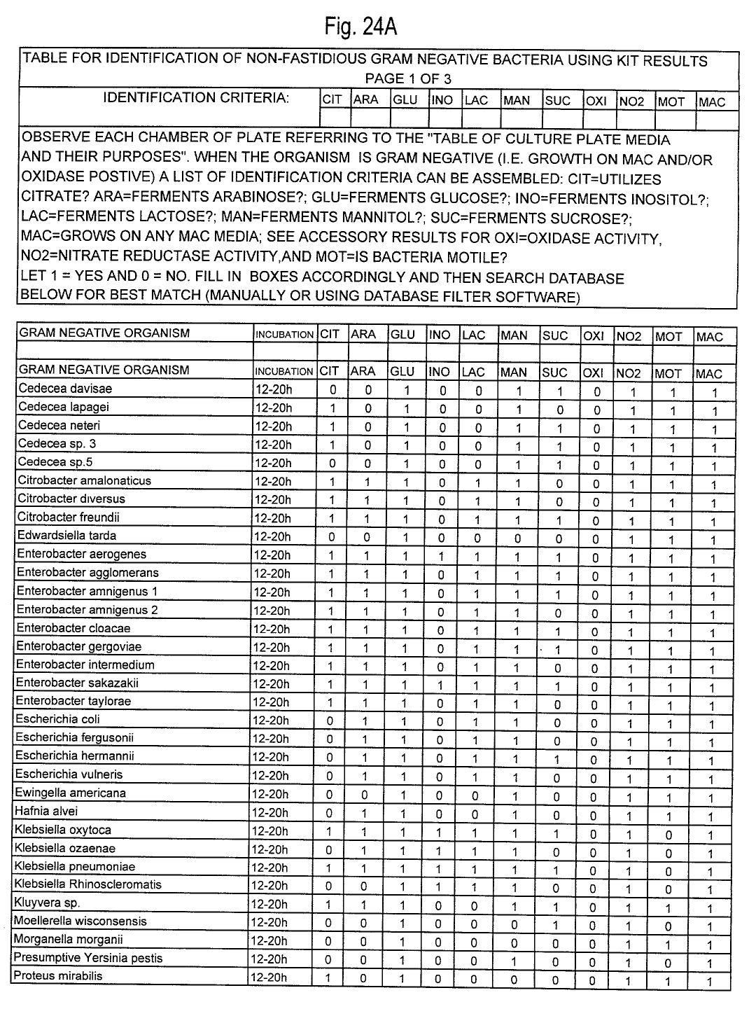

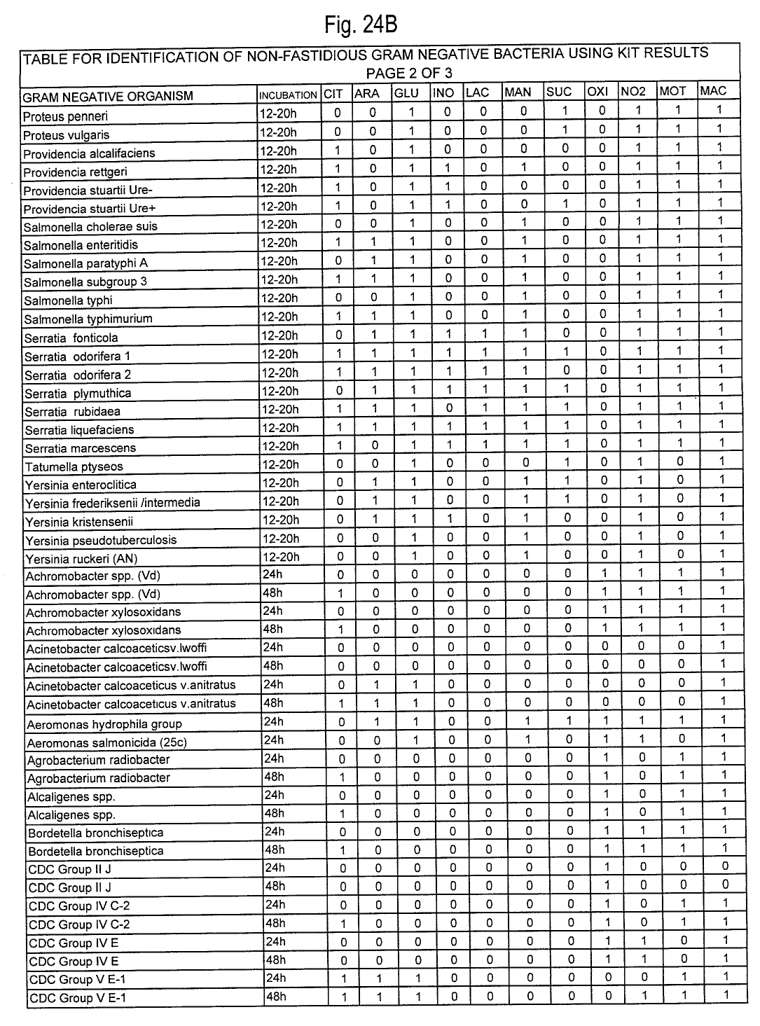

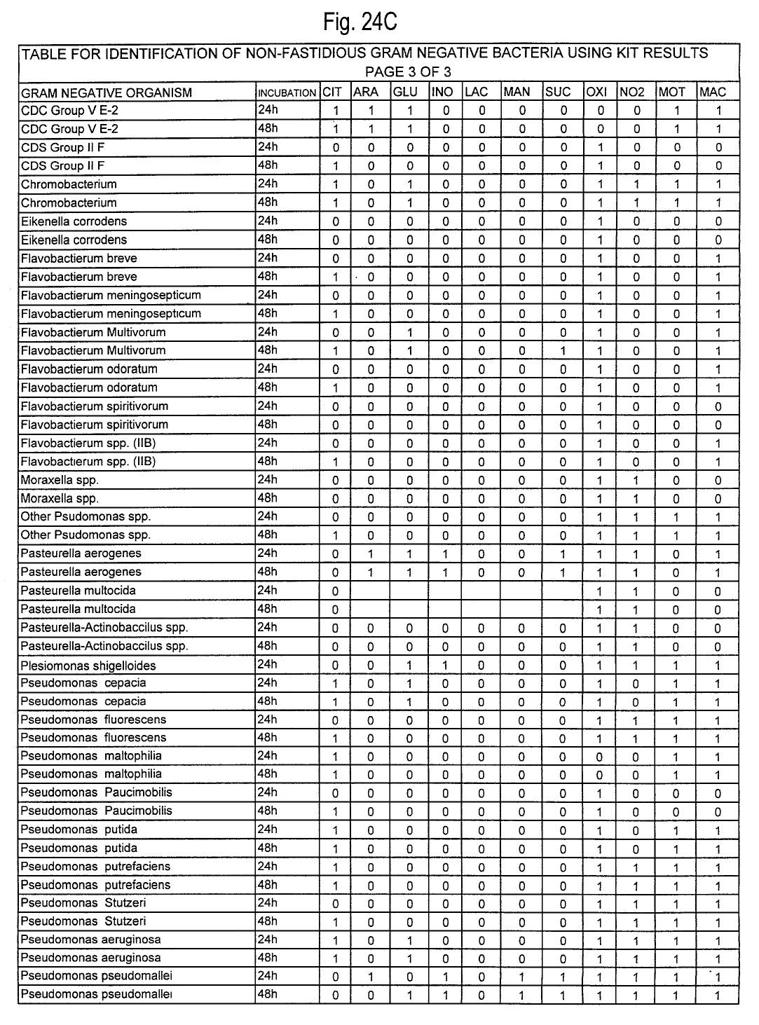

FIGS. 24A to 24C shows a table for identifying non-fastidious gram-negative bacteria using kit results.

FIG. 25 shows a blank kit plate before addition of microorganisms.

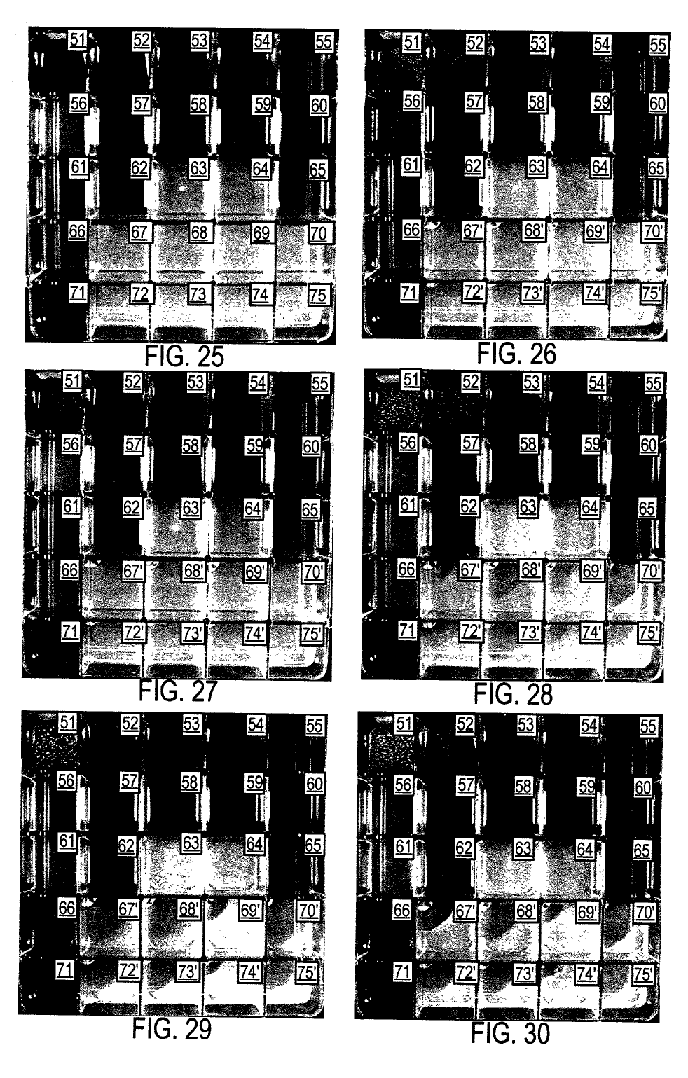

FIG. 26 shows an example of a kit plate incubated for 16 hrs following inoculation with the gram-positive organism Enterococcus faecalis ATCC 29212

FIG. 27 shows an example of the preferred embodiment kit plate incubated for 20 hrs following inoculation with the gram-positive organism Streptococcus pyogenes ATCC 19615

FIG. 28 shows an example of the preferred embodiment kit plate incubated for 16 hrs following inoculation with the gram-positive organism Staph. epidermidis ATCC 12228

FIG. 29 shows an example of the preferred embodiment kit plate incubated for 16 hrs following inoculation with the gram-positive organism Staph. aureus ATCC 25923

FIG. 30 shows an example of the preferred embodiment kit plate incubated for 16 hrs following inoculation with the gram-positive organism Staph. aureus ATCC 29213

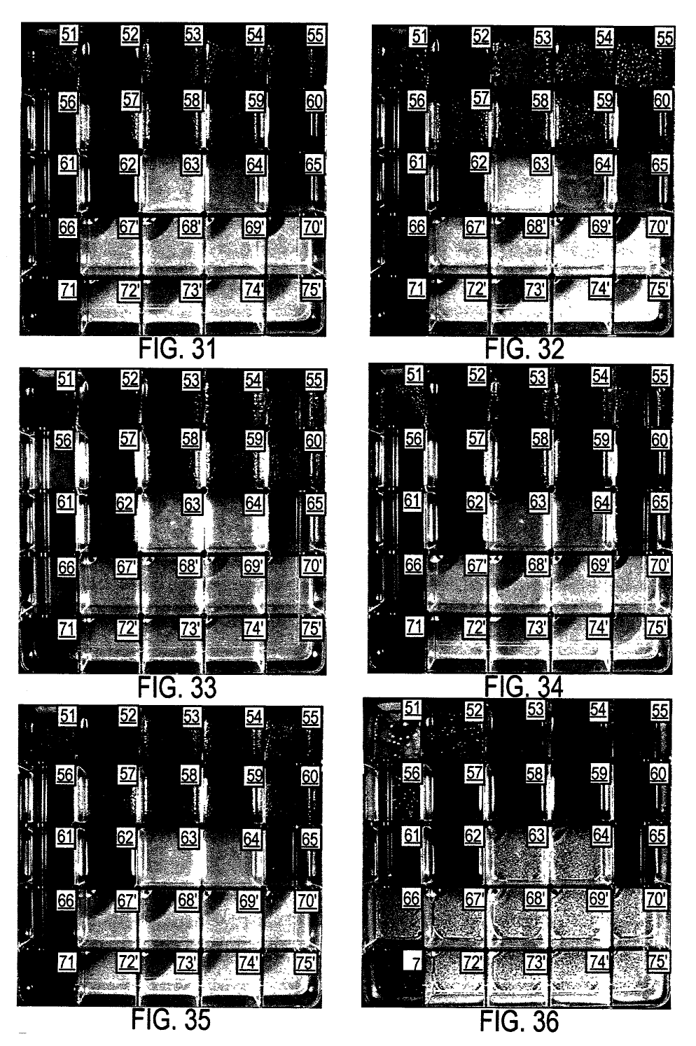

FIG. 31 shows an example of the preferred embodiment kit plate incubated for 16 hrs following inoculation with the gram-negative organism E. coli ATCC 25922

FIG. 32 shows an example of the preferred embodiment kit plate incubated for 16 hrs following inoculation with the gram-negative organism Klebsiella pneumoniae ATCC 13883

FIG. 33 shows an example of the preferred embodiment kit plate incubated for 24 hrs following inoculation with the gram-negative organism Pseudomonas aeruginosa ATCC 27853

FIG. 34 shows an example of the preferred embodiment kit plate incubated for 20 hrs following inoculation with the gram-negative organism Proteus vulgaris ATCC 13315

FIG. 35 shows an example of the preferred embodiment kit plate incubated for 16 hrs following inoculation with the gram-negative organism Salmonella typhimurium ATCC 14028

FIG. 36 shows an example of the preferred embodiment kit plate incubated for 48 hrs following inoculation with the fungus Candida albicans ATCC 14053

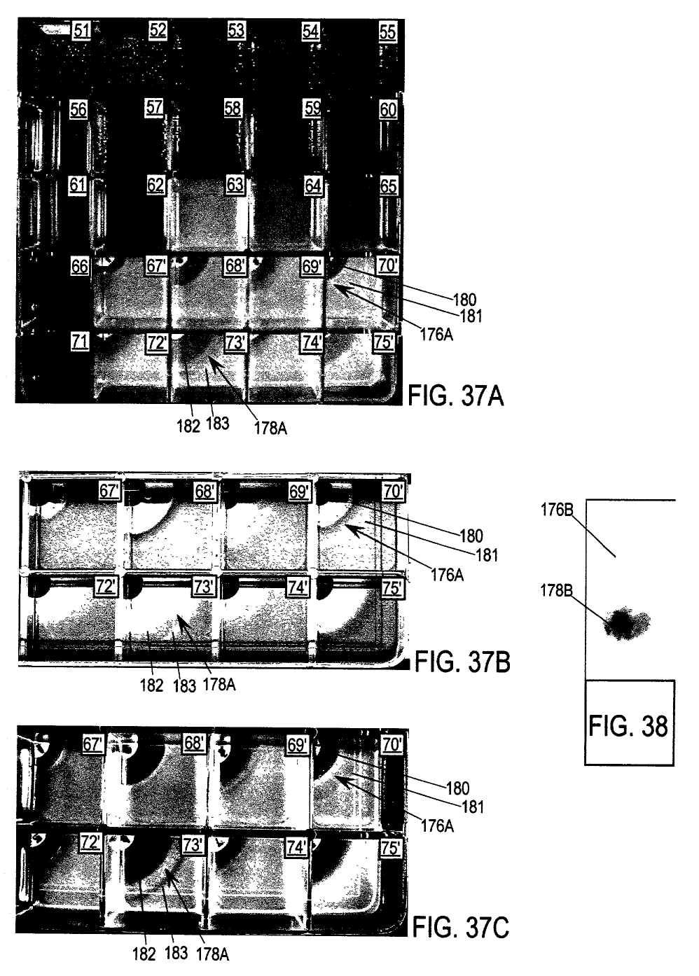

FIG. 37 A shows an example of an incubated kit plate consisting of a mixture of E. coli and Staphylococcus aureus.

FIGS. 37A to 37C illustrates the microorganisms in FIG. 37A being differentiated on the preferred embodiment kit plate in terms of ID as well as AST.

FIG. 38 shows a gram stained slide of the two microorganisms from FIG. 37 separated by their difference in AST to two antimicrobial agents.

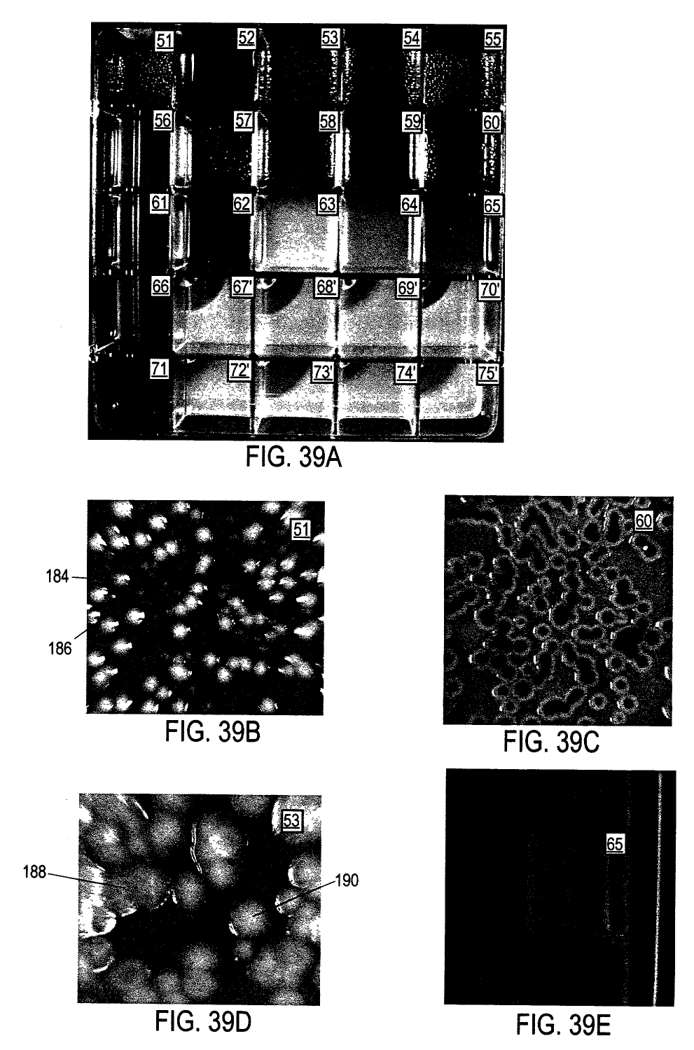

FIGS. 39A to 39E illustrates two microorganisms being differentiated on the preferred embodiment kit plate in terms of differences in medium selectivity, fermentation and production of fluorescent product formation.

### REFERENCE NUMERALS

51 Blood agar chamber

52 Azide blood agar chamber

53 Lactose MacConkey agar chamber

54 Glucose MacConkey agar chamber

55 Mannitol MacConkey agar chamber

56 Bile esculin azide agar chamber

57 Inositol MacConkey agar chamber

58 Sucrose MacConkey agar chamber

59 Arabinose MacConkey agar chamber

60 Hektoen enteric agar chamber

61 Mannitol salt agar chamber

62 Simmons citrate agar chamber

63 Pseudomonas agar F chamber

64 Pseudomonas agar P chamber

65 MUG MacConkey agar chamber

66 Tellurite Glycine agar chamber

67 Mueller Hinton agar chamber

67′ Mueller Hinton agar plus antimicrobial Ampicillin chamber

68 Mueller Hinton agar chamber

68′ Mueller Hinton agar plus antimicrobial Amoxicillin/Clavulanic 69 acid (Augmentin) chamber

69′ Mueller Hinton agar chamber

70 Mueller Hinton agar plus antimicrobial Amikacin chamber

70 Mueller Hinton agar chamber

70′ Mueller Hinton agar plus antimicrobial Cephalothin chamber

71 Littman oxgall agar chamber

72 Mueller Hinton agar chamber

72′ Mueller Hinton agar plus antimicrobial Doxycycline chamber

73 Mueller Hinton agar chamber

73′ Mueller Hinton agar plus antimicrobial Enrofloxicin chamber

74 Mueller Hinton agar chamber

74′ Mueller Hinton agar plus antimicrobial Gentamicin chamber

75′ Mueller Hinton agar chamber

75′ Mueller Hinton agar plus antimicrobial Septra chamber

76 One forth of a Kirby-Bauer AST disk (antimicrobial disk-quarter)

77 Multi-chambered kit plate with antimicrobial disk-quarters

77′ Multi-chambered kit plate cut-away for illustrative purpose only

78 Multi-chambered kit plate Lid

80 Multi-chambered kit plate before antimicrobial disk-quarters are added

82 Brain heart infusion broth (BHIB)

84 Culture swab containing an initial specimen sample

85 Culture swab containing a sample from a suspension of an isolated organism

86 Tear-away lid

88 Bottle stopper

90 Inoculated incubated Brain heart infusion broth (IIBHIB)

92 McFarland turbidity standard, 0

94 McFarland turbidity standard, 0.5

96 0.5 McFarland equivalent dilution of IIBHIB

98 McFarland turbidity standard, 1

100 Sterile transfer pipette

102A Antimicrobial disk-quarter placement device, charge position

102B Antimicrobial disk-quarter placement device, discharge position

104 Quilter's pin

106 Push-off slider

108 Antimicrobial storage container

110 Antimicrobial set of unique disk-quarters for AST

111 Culture transport system

112 Agar plate for primary isolation (Blood agar for example)

113 Cap for culture transport system

116 Inoculum applied to agar plate

118 Inoculating loop

120 Streaking tracks

122 Standard Susceptibility Plate (Mueller Hinton agar for example)

124 AST disk (prior art) with impregnated antimicrobial agent

125 Microorganism growth on a Standard Susceptibility Plate

126 Margin or interface between growing 125 and inhibited 127 microorganisms

127 Region of inhibited bacteria growth due to antimicrobial agent

128 Diffusion of antimicrobial agent into agar from impregnated antimicrobial disk

129T Right thumb placement on top of disk-quarter placement device

129F Right index finger placement on top of disk-quarter placement device

130T Left thumb placement on slider of disk-quarter placement device

130F Left index finger placement on slider of disk-quarter placement device

132 Culture broth for anaerobes (Thioglycolate broth)

134 Inoculated incubated thioglycolate broth

135 Incubator

136 Sterile diluent

138 Intermediate dilution from 0.5 McFarland equivalent (1 to 20)

140 Final dilution from the intermediate dilution (1 to 50) for 1 to 1000 of 0.5 McFarland equiv.

141 Zone of inhibition (prior art) measured as diameter of the region of non-growth

142 Zone of inhibition measured as radius in square chamber with antimicrobial disk-quarter

144 Chamber with AST medium

145 Cap for reagent vial

146 Griess reagent sulfanilamide

148 Griess reagent N-(1-napthyl) ethylenediamine

150 Phosphoric acid diluent

152 Griess working reagent

154 Positive griess reaction after adding broth

156 Negative griess reaction after adding broth

158 Zinc dust added to 156

160 Positive griess reaction after zinc

162 Negative griess reaction after zinc

164 Applicator for oxidase test

166 Oxidase test paper

168 Water to hydrate oxidase paper

170 Sample of microorganism colony applied to test paper

172 Positive oxidase test

174 Negative oxidase test

176A E. coli growth region showing resistance to Cephalothin in mixed culture with Staph. aureus

176B Gram stain of E. coli region

178A Staph. aureus growth region showing resistance to Enrofloxicin in mixed culture with E. coli

178B Gram stain of Staph. aureus region

180 Margin of E. coli with the antimicrobial Cephalothin

181 Margin of Staph. aureus with the antimicrobial Cephalothin

182 Margin of Staph. aureus with the antimicrobial Enrofloxicin

183 Margin of E. coli with the antimicrobial Enrofloxicin

184 E. coli colony on blood agar

186 Salmonella typhimurium colony on blood agar

188 Lactose fermenting E. coli colony in mixed culture of E. coli and Salmonella

190 Non-lactose fermenting Salmonella in mixed culture of E. coli and Salmonella

### Preferred Embodiment

The following section lists the static physical structure and components of the preferred embodiment. An overview (summary) of the principle components of the kit is illustrated on page 1/18 of the figures section. FIG. 1 shows a perspective view of the preferred embodiment of a portion of the present invention, an ethylene oxide sterilized polypropylene multi-chambered kit plate 77 with square test chambers. Eight of the test chambers (67′-70′ and 72′-75′) show antimicrobial disk-quarters 76 in the corners of the test chambers. Quartering Standard Kirby-Bauer AST disks using a plastic jig and a razor blade is one way of preparing the disk-quarters. Labeling the disk-quarters is currently done by hand. Media in the test chambers are at a depth of 4 mm, which occupies a volume of 1.6 milliliters and a surface area of 4 cm squared. Different types of diagnostic agar-based media are used in the test chambers of the kit plate: Blood agar 51 (Enriched); Simmons citrate 62 (Differential); Azide blood agar 52 (Selective); Lactose MacConkey agar 53, Glucose MacConkey agar 54, Mannitol MacConkey agar 55, Inositol MacConkey agar 57, Sucrose MacConkey agar 58, Arabinose MacConkey agar 59, Hektoen enteric agar 60, Mannitol salt agar 61, Pseudomonas agar F 63, Pseudomonas agar P 64, and MUG MacConkey agar 65, (Differential-Selective); Bile esculin azide agar 56, Tellurite Glycine agar 66, Littman oxgall agar 71, and Mueller Hinton agar 67-70 and 72-75 (Single purpose). FIG. 2 shows a color view of the preferred embodiment kit plate before antimicrobial disk-quarters are applied to the kit plate 80. Lid 78 is also shown.

FIG. 3 illustrates Brain Heart infusion broth (BHIB) 82 and a specimen containing culture swab 84. The breakaway cap 86 and stopper 88 are associated with the broth 82 container. Incubator 135 and Inoculated incubated Brain Heart infusion broth (IIBHIB) 90 are also illustrated.

FIG. 4 illustrates a dilution 96 of the Inoculated incubated Brain Heart infusion 90, to a concentration that is equivalent to a 0.5 McFarland turbidity standard 94. Other standards are zero McFarland turbidity standard 92 and 1 McFarland turbidity standard 98.

FIG. 5 illustrates the preferred embodiment kit plate 80 ready to receive dilution 96, one of two dilutions used for kit plate inoculation. Disposable sterile pipette 100 is also shown.

FIG. 6 shows a device 102A shown in the charging position used in the placement of antimicrobial disk-quarters 110. The device comprises two parts: a quilter's pin 104 and a push-off slider 106 made from a small pipette tip. The antimicrobial storage container 108 is also shown. FIG. 18 illustrates the placing device in the discharge position 102B

FIG. 37A illustrates an incubated kit plate containing two microorganisms and will be described in more detail later on.

FIG. 15 illustrates Thioglycolate broth (Thio) 132 and a culture swab containing an initial specimen sample 84. The breakaway cap 86 and stopper 88 are associated with the broth 132 container. Incubator 135 and Inoculated incubated Thioglycolate broth 134 is also illustrated.

FIGS. 16A-16D illustrates components for preparation of dilutions from the Inoculated incubated Brain heart infusion broth (IIBHIB) 90: pipette 100, sterile diluent 136, cap 142, McFarland turbidity standards: “0” Standard 92, 0.5 Standard 94, and “1.0” Standard 98 and additional diluents 138 and 140.

FIG. 17 illustrates the preferred embodiment kit plate 80, ready to receive dilution 140, second of two dilutions. Also shown is a disposable sterile pipette 100.

FIG. 18 shows a cut away view of a multi-chambered kit plate 77′ with antimicrobial disk-quarters 76 placed in Mueller Hinton containing test chambers (67′, 68′, 69′, 70′, 72′, 73′, 74′, 75′). The antimicrobial disk-quarter placement device is at discharge position 102B. Also shown is device in charge position 102A, a set of antimicrobial disk-quarters 110 (representing 8 different antimicrobial agents), container 108, and finger and thumb placement positions for manipulating the placement device: position 129T, position 129F, position 131T, and position 131F.

FIGS. 19A-19B illustrates the equivalence between prior art and an AST chamber 144 from the kit multi-chambered kit plate 77. The principle of the standard Kirby-Bauer disk-diffusion AST test is illustrated in FIG. 19A (review FIGS. 12C-12D). This illustration shows how the preferred embodiment system (FIG. 19B, zone radius 142) measures exactly one-half of the measurement of zone diameter 141, the prior art standard method. The zone of inhibition radius measurement 142 is measured from the corner of the chamber containing disk-quarter 76 to margin 126. The interface between growing microorganisms 125 and inhibited microorganisms 127.

FIGS. 20A-20D shows the components of a modified nitrate reductase assay: reagent vial lid 145, Griess reagent sulfanilamide 146, Griess reagent N-(1-napthyl) ethylenediamine 148, and phosphoric acid diluent 150. When the three are combined, they make up the Griess working reagent 152. FIG. 20C illustrates a positive griess reaction 154 or a negative griess reaction 156. FIG. 20D illustrates the addition of zinc powder 158 and either a positive griess reaction after zinc 160 or a negative griess reaction after zinc 162.

FIGS. 21A-21C shows the components of a cytochrome oxidase assay: applicator 164, oxidase test paper 166, and water 168. FIG. 21B shows sample 170 addition and FIG. 21C illustrates a positive oxidase test 172 or a negative oxidase test 174.

FIG. 22 shows a table of the preferred embodiment media components of the multi-chambered kit plate and associated reference numerals as described earlier this section.

FIG. 23 illustrates the preferred embodiment kit plate media layout with the associated reference numerals as described previously this section.

FIGS. 24A to 24C shows the table used for ID of non-fastidious gram negative bacteria using the results from the incubated kit plate, the nitrate reductase test, the oxidase test, and morphology and motility observations.

The following figures (FIG. 25-FIG. 36) show results of incubations with a number of different microorganisms. The OPERATION OF INVENTION section will describe the details of ID and AST for each kit plate.

FIG. 25 shows preferred embodiment blank kit plate before addition of microorganisms.

FIG. 26 shows an example of preferred embodiment kit plate inoculated and incubated for 16 hrs with the gram-positive organism Enterococcus faecalis ATCC 29212

FIG. 27 shows an example of preferred embodiment kit plate inoculated and incubated for 20 hrs with the gram-positive organism Streptococcus pyogenes ATCC 19615

FIG. 28 shows an example of preferred embodiment kit plate inoculated and incubated for 16 hrs with the gram-positive organism Staph. epidermidis ATCC 12228

FIG. 29 shows an example of preferred embodiment kit plate inoculated and incubated for 16 hrs with the gram-positive organism Staph. aureus ATCC 25923

FIG. 30 shows an example of preferred embodiment kit plate inoculated and incubated for 16 hrs with the gram-positive organism Staph. aureus ATCC 29213

FIG. 31 shows an example of preferred embodiment kit plate inoculated and incubated for 16 hrs with the gram-negative organism E. coli ATCC 25922

FIG. 32 shows an example of preferred embodiment kit plate inoculated and incubated for 16 hrs with the gram-negative organism Klebsiella pneumoniae ATCC 13883

FIG. 33 shows an example of preferred embodiment kit plate inoculated and incubated for 24 hrs with the gram-negative organism Pseudomonas aeruginosa ATCC 27853

FIG. 34 shows an example of preferred embodiment kit plate inoculated and incubated for 20 hrs with the gram-negative organism Proteus vulgaris ATCC 13315

FIG. 35 shows an example of preferred embodiment kit plate inoculated and incubated for 16 hrs with the gram-negative organism Salmonella typhimurium ATCC 14028

FIG. 36 shows an example of preferred embodiment kit plate inoculated and incubated for 48 hrs with the fungus Candida albicans ATCC 14053

FIGS. 37A to 37C illustrates the growth of two microorganisms, Staph. aureus and E. coli, on the same preferred embodiment kit plate. FIGS. 37B and 37C are two different views of an enlargement of the AST test chambers (67′-70′ and 72′-75′). FIG. 37B viewed with back lighting. FIG. 37C viewed with front lighting. Two AST test chambers are featured in these figures: Mueller Hinton agar plus antimicrobial Cephalothin chamber 70′ and Mueller Hinton agar plus antimicrobial Enrofloxicin chamber 73′. Two margins are observable in each chamber: E coli & cephalothin margin 180 and Staph. aureus & cephalothin margin 181 for Cephalothin chamber 70′; and Staph. aureus & Enrofloxicin margin 182 and E. coli & Enrofloxicin margin 183 for Enrofloxicin chamber 73′. The region 176A is between the E. coli margin 180 and Staph. aureus margin 181 in Cephalothin chamber 70′. The region 178A is between the Staph. aureus margin 182 and the E. coli margin 183 in Enrofloxicin chamber 73′. Illustrated in FIG. 38 are two gram stains 176B and 178B of a sample from regions 176A and 178A respectively.

FIGS. 39A to 39E is an example of a preferred embodiment kit plate inoculated with a mixture of two microorganisms E. coli and Salmonella typhimurium. These sets of figures illustrate the utility of the method and kit when several microorganism types are present. FIG. 39A shows the preferred embodiment kit plate and the result of growth and biochemistry of the organisms on the various media. FIG. 39B is a 10× magnification view of Blood agar chamber 51. The view shows two different types of colonies based on size: An E. coli colony 184 and a Salmonella typhimurium colony 186. FIG. 39C is a 10× magnification view of Hektoen enteric agar chamber 60, illustrating the result of the growth of Salmonella upon the agar. FIG. 39D shows two colony types in the Lactose MacConkey chamber 53, differentiated in terms of their ability to ferment lactose. The pink centered lactose fermenting, E. coli 188 is contrasted to the non-lactose fermenting Salmonella 190. FIG. 39E illustrates the fluorescence in the MUG MacConkey chamber 65, due to the action of a specific enzyme found in E. coli.

### Operation-Preferred Embodiment

A description of the manner of using the preferred embodiment of the kit is in this section. FIG. 1 as indicated in the description of the preferred embodiment is comprised of Enriched, Differential, Selective, Differential-Selective and Single purpose media. The formulations and methods of preparation of the various media used in this kit are available from the Eleventh edition of the Difco Manual. One modification to those formulas may comprise the incorporation of iota carrageenan to the agar-based media for reducing the watering out of the hydrocolloid agar gel (syneresis) as well as increasing stability and shelf life. I however do not wish to be bound by this observation. The multi-chambered kit plate 80 and associated lid 78 are packed under nitrogen atmosphere in a low oxygen-permeable sealed bag to extend the shelf life.

The process of kit plate media preparation follows standard practices of sterile technique. Envisioned but not illustrated is a process that could be used to produce the kit plates in an efficient fashion. The system conceptually would comprise a temperate regulated box with lid, large enough to hold the individual kit plate chamber medium vessels at 50° C. plus. The distribution of that media to the test chambers of the multi-chambered kit plate would be accomplished by using a dispensing pump able to dispense the correct amount of media into each test chamber. The pump would drive a multi-channeled pump head with the same number of channels as the number of test chambers in the kit plate allowing for a relatively simple method for manufacturing the multi-chambered kit plates.

A description of the diagnostic usefulness of each of the medium of the multi-chambered kit plate is as follows:

Blood agar 51 is used in the isolation of a wide variety of microorganisms. All non-fastidious gram-negative and gram-positive organisms will grow on this medium. The majority of the aerobic gram-positive and gram-negative bacterial pathogens of domestic animals and man will grow on blood agar when incubated in air at 35° C. Blood agar also allows for determination of hemolytic patterns. The hemolytic patterns adjacent to bacterial colonies are classified as non-hemolytic (gamma hemolysis), complete (beta-hemolysis), and partial (alpha-hemolytic).

Bile esculin azide agar 56 is used for isolating, differentiating and presumptively identifying group D streptococcus and Enterococcus. These organisms cause the formation of a dark brown or black complex in the agar.

Mannitol salt agar 61 allows staphylococci to grow while the growth of most other bacteria is inhibited.

Tellurite Glycine agar 66 permits the isolation of coagulase positive staphylococci whereas coagulase negative staphylococci and other bacteria are completely inhibited. Coagulase positive staphylococci reduce tellurite and produce black colonies.

Littman oxgall agar 71 is used for the isolation of fungi and is suitable for growth of pathogenic fungi. Incubation is for several days. Molds and yeasts form non-spreading, discrete colonies.

Azide blood agar 52 is used in the isolation of gram-positive organisms from clinical and non-clinical specimens. Azide suppresses the growth of gram-negative bacteria and is useful in determining hemolytic reactions.

Simmons citrate agar 62 is used in the ID of gram-negative organisms that are able to metabolize citrate. The citrate-utilizing organisms grow luxuriantly and the medium becomes alkalinized and changes from its initial green to deep blue.

Pseudomonas agar F 64 is used for differentiating Pseudomonas aeruginosa from other pseudomonads based on fluorescein production and is visible with UV lamp at 365 nm.

Pseudomonas agar P 64 is used for differentiating Pseudomonas aeruginosa from other pseudomonads based on the production of pyocyanin, a non-fluorescent blue pigment.

Hektoen enteric agar 60 is used to isolate and differentiate Salmonella. Colonies are greenish blue, with black centers.

MUG MAC 65 is a MacConkey agar with lactose plus an added substrate 4-methylumbelliferyl-b-D-glucuronide (MUG). MUG becomes fluorescent when E. coli is present. The E. coli beta-glucuronidase enzyme cleaves the colorless MUG to a fluorescent product detected with UV light at 365 nm.

MacConkey agar, which contains bile salts, is a selective media for the majority of gram-negative pathogens. The media inhibits gram-positive bacteria and a few gram-negative pathogens. Almost 100% of the genera from the family Enterobacteriaceae (all being gram negative), and nearly 80% of other gram-negative genera grow on MacConkey agar. The preferred embodiment following MAC media contain six different sugars to allow for ID of microorganisms based on their fermentation patterns:

Lactose MAC 53 is MacConkey agar with lactose, a selective and differential medium for growing gram-negative bacilli. Lactose fermenting strains grow as red or pink colonies.

Glucose MAC 54 is MacConkey agar with glucose, a selective and differential medium for growing gram-negative bacilli. Glucose fermenting strains grow as red or pink colonies.

Mannitol MAC 55 is MacConkey agar with mannitol, a selective and differential medium for growing gram-negative bacilli. Mannitol fermenting strains grow as red or pink colonies.

Inositol MAC 57 is MacConkey agar with inositol, a selective and differential medium for growing gram-negative bacilli. Inositol fermenting strains grow as red or pink colonies.

Sucrose MAC 58 is MacConkey agar with sucrose, a selective and differential medium for growing gram-negative bacilli. Sucrose fermenting strains grow as red or pink colonies.

Arabinose MAC 59 is MacConkey agar with arabinose, a selective and differential medium for growing gram-negative bacilli. Arabinose fermenting strains grow as red-pink colonies.

Mueller Hinton agar (67-70, 72-75) is considered the best media for routine AST of non-fastidious bacteria. Eight test chambers are set-aside for this purpose.

FIG. 3 illustrates a crucial part of the method and kit. Instead of the 24 hr prior art method of streaking to isolate individual organisms, a short 4 to 8 hr incubation in Brain-Heart Infusion broth is utilized. A swab specimen 84 inoculates the broth for growth to a stationary phase 90 in incubator 135. Thioglycolate broth is also included for growth of potential anaerobic microorganisms 134.

The isolation of the microorganisms is concurrent with a 12 to 20 hr. ID testing on the kit plate. Specifically, isolated colonies become visible in the top two rows of the kit plate. These rows received the higher dilution (lower concentration) of microorganism(s) 140. The bottom three rows of the kit plate seeded with a higher concentration of microorganisms 96 performs the AST in addition to other tests as described below.

FIGS. 16A, 16B, 16C, and 16D show the process of preparing the two dilutions (dilution 96 and dilution 140) from the IIBHIB 90 for the kit plate inoculation steps as shown in FIG. 5 and FIG. 17. This accomplishes the same thing as the streaking out on a microbiological agar plate as described in the prior art but is much simpler. Especially where there are multiple test chambers involved. The result is that in the top two rows of the kit plate the microorganisms grow up into individual colonies for the purpose of ID. The more concentrated 0.5 McFarland equivalent dilution 138 produces a lawn of microorganisms for the AST test chambers and the other test chambers in the bottom three rows. For the initial kit plate inoculation, a sterile-forty microliter/drop-pipette 100 removes a portion of the grown up (usually to stationary phase) microorganisms (IIBHIB 90). Five drops into sterile diluent 136 results in a 1 to 11 dilution. The dilution 96 created is generally close to a 0.5 McFarland turbidity standard as shown in FIG. 16C. Dilution 96 and standard 94 are compared against a black lined background and adjustments made so dilution 96 will be close in turbidity to standard 94. The 0.5 McFarland equivalent dilution 96 is in turn diluted 1 to 1000 by making a 1 to 20 and then a 1 to 50 dilution that becomes the 1 to 1000 dilution 140.

FIG. 5 shows an addition of dilution 96 to the first of fifteen test chambers of the bottom three rows of the kit plate. Eighty milliliters of this dilution is added per chamber. FIG. 17 illustrates the first chamber of the top two rows inoculated using dilution 140. Eighty micro liters of dilution 140 is added per chamber to the ten upper test chambers. Following the additions of the 2 dilutions, the microorganisms are spread out on the surface of the media by briefly shaking the kit plate in a back and forth motion in both directions. The excess liquid is removed by tapping the kit plate upside down into the lid containing an absorbent tissue. The kit plate is allowed to air-dry for approximately 10 minutes before the addition of antimicrobial disk-quarters 76.

The method design and placement of antimicrobial agents on the AST portion of the kit plate is a novel and unique modification of the standard Kirby-Bauer disk-diffusion method for AST. FIGS. 19A-19B illustrates the equivalence between prior art (FIG. 19A) and an AST chamber 144 from the kit plate. One forth of an AST disk 124, called a disk-quarter 76, is placed in the corner of chamber 144 giving exactly the same result multiplied by 2 as the standard disk-diffusion method (inhibition radius 142×2=inhibition diameter 141). FIG. 18 illustrates the placement of disk-quarters 76 into the test chambers using placement device 102. The thumbs and index fingers of both hands hold the device as shown by the position 129T(right thumb), 129F(right-index finger), 131T(left thumb), and 131F(left-index finger). The hands can be switched if desired. The disk-quarter 76 is picked up with the placement device 102A using a piercing motion into the disk-quarter. The disk-quarter 76 is rotated and placed in the corner of the test chamber. The disk-quarter is then removed by pushing off with the slider as shown by device discharge position 102B.

The kit also includes a modified Nitrate Reductase determination system FIGS. 20A-20D as well as a Cytochrome oxidase test as shown in FIGS. 21A-21C. Anyone skilled in the art will be able to perform these tests, results of which will add additional pieces to the ID puzzle. See FIGS. 24A-24C below for a mechanism of ID. The Nitrate Reductase is determined on the IIBHIB 90 whereas the Oxidase test is run on the individual colonies.

FIG. 22 and FIG. 23 show the preferred embodiment of the kit plate layout and a listing of the culture kit plate media and their purposes.

FIGS. 24A to 24C shows a database table for identifying non-fastidious gram-negative bacteria using kit results. This database is supplied with the kit and can be searched. After filling in the criteria, search the database for the best match. In some cases, the result is unique. Other cases result in several presumptive choices. However, if other criteria are included, such as colony morphology or cellular morphology, a more definitive ID is possible. Additionally several of the kit plate medium allow for definitive ID such as Hektoen enteric agar for Salmonella, MUG MAC for E. coli and Pseudomonas agar F for the expression of fluorescein in identifying Pseudomonas aeruginosa. When the microorganisms form colonies on the MAC media and/or are oxidase positive, the criteria that are utilized for the above database include the following: Citrate utilization; arabinose fermentation; glucose fermentation; inositol fermentation; lactose fermentation; mannitol fermentation; sucrose fermentation; growth on MacConkey based agar; oxidase activity; nitrate reduction; and. microorganism motility. All but the last three criteria are obtained from an analysis of the cultured multi-chambered kit plate. The oxidase test and nitrate reductase test are done separately as mentioned above. A discussion of bacterial motility takes place in the next paragraph. The criteria are keyed in as “0” for a negative result and “1” for a positive result and then the database is filtered using a spreadsheet software program. Alternatively, the database can be manually searched.

Motility of bacteria is an important characteristic in the ID of unknown bacteria. To test for bacteria, a drop of incubation broth is placed on a clean glass slide, a cover slip is added, and the cells are viewed directly for motility. Three types of motion are seen under a microscope: (1) Brownian motion, which is the result of the bombardment of water molecules, (2) Fluid-movement that is due to capillary action, and (3) Motility, which is self-propulsion. The difference between Brownian movement and motility is that motile bacteria move through the liquid whereas in Brownian motion the bacteria just vibrate.

The following figures (FIG. 25-FIG. 36) show results of incubations with a number of different single microorganisms. FIGS. 37A-37C, FIG. 38, and FIGS. 39A-39E illustrate results where more than one organism exists on the same kit plate. This situation highlights the power of the Kit and method when two microorganisms are present from the same specimen.

FIG. 25 shows a blank kit plate with media before the addition of microorganisms. This figure provides the initial baseline for appearance and color reactions that take place in the kit plate with various organisms.

FIG. 26 shows an example of preferred embodiment kit plate inoculated and incubated for 16 hrs at 35° C. with the gram-positive organism Enterococcus faecalis, ATCC 29212. Blood agar chamber 51 supports the growth of this organism as well as all non-fastidious gram-negative and gram-positive organisms. Shown are non-hemolytic colonies. Azide blood agar chamber 52 also supports the growth of this organism and is non-hemolytic. Gram-negative organisms do not grow on this medium. Bile esculin azide agar chamber 56 shows the formation of a dark brown or black complex in the agar, which is unique to the growth of group D streptococcus and Enterococcus organisms. Test chambers (67′-70′, 72′-75′) are the Mueller Hinton AST test chambers containing eight different antimicrobial agents (see the Drawings-reference numerals for the antimicrobial used). The pattern of AST is only faintly evident from this depiction and will not be discussed here.

FIG. 27 shows an example of preferred embodiment kit plate inoculated and incubated for 20 hrs with the gram-positive organism Streptococcus pyogenes ATCC 19615. Blood agar chamber 51 reveals beta-hemolytic small colonies. Azide blood agar chamber 52 also supports growth.

FIG. 28 shows an example of preferred embodiment kit plate inoculated and incubated for 16 hrs with the gram-positive organism Staph. epidermidis ATCC 12228. Note the growth in the Blood agar chamber 51 and in the Azide blood agar chamber 52. Note the growth in the Mannitol salt agar 61. All staphylococci will grow in this medium while the growth of most other bacteria is inhibited. Note also the neutral to slightly alkaline color due to the phenol red pH indicator. This shows that the mannitol is not fermented. Fermentation would result in acid products turning the medium yellow. The tellurite Glycine agar 66 also shows no growth. The Staph. epidermidis is coagulase negative organism and that media only permits the growth of coagulase positive Staphylococcus. The coagulase-negative staph. and other bacteria are completely inhibited on this medium. The coagulase-positive staph. reduce tellurite and produce black colonies when present. Pseudomonas agar F chamber 63 and Pseudomonas agar P chamber 64, support growth of the Staph. epidermidis but no pigment is produced.

Note the different antimicrobial susceptibilities in FIG. 28, test chambers (67′-70′, 72′-75′). To illustrate how AST is performed, look at, for example, FIG. 28 chamber 68′, which contains the antimicrobial agent Amoxicillin/Clavulanic acid (Clavamox or Augmentin). Then refer to FIG. 19B which show a schema of a typical endpoint antimicrobial-containing Mueller Hinton chamber. A measurement 142, in millimeters using ruler or caliper, is made from the disk-quarter 76 chamber corner to the margin 126 (the interface between the growing 125 and inhibited 127 microorganisms). To then determine if the microorganisms are Resistant, Intermediate, or Susceptible to the antimicrobial agent, match the measured value to the value listed in the Modified Interpretive Standards Table (see below).

MODIFIED INTERPRETIVE STANDARDS TABLE:

| | Distance from antibiotic corner of | | |
|---|---|---|---|
| | chamber to growth margin (mm) | | |
| Antimicrobial Agent | Resistant | Intermediate | Susceptible |
| Ampicillin when testing gram negative | ≦5.5 | 6-6.5 | ≧7 |
| enteric organisms and enterococci | | | |
| Ampicillin when testing staphylococci and | ≦10 | 10.5-14 | ≧14.5 |
| penicillin G susceptible microorganisms | | | |
| Clavamox when testing gram negative | ≦6.5 | 7-8.5 | ≧9 |
| enteric organisms and enterococci | | | |
| Clavamox when testing staphylococci and | ≦9.5 | — | ≧10 |
| penicillin G susceptible microorganisms | | | |
| Amikacin | ≦7 | 7.5-8 | ≧8.5 |
| Cephalothin (Keflex) | ≦7 | 7.5-8.5 | ≧9 |
| Doxycycline | ≦6 | 6.5-7.5 | ≧8 |
| Enrofloxicin (Baytril) | <8 | 8-10 | >10 |
| Gentamicin | ≦5 | 5.5-6.5 | ≧7 |
| Trimethoprim-sulfamethoxazole (Septra) | ≦5 | 5.5-7.5 | ≧8 |

FIG. 29 and FIG. 30 show examples of two preferred embodiment kit plates incubated for 16 hrs following inoculation with Staph. aureus ATCC 25923 and Staph. aureus ATCC 29213 respectively. One primary difference between these two strains is their difference in AST to Ampicillin. Note Ampicillin test chamber 67′ in FIG. 29 and FIG. 30. Note the Tellurite Glycine agar test chamber 66, illustrating the growth of Staph. aureus (a coagulase-positive Staph.) and reduction of the tellurite. Contrast this result to FIG. 28 test chamber 66. Also, note the growth in FIG. 29 and FIG. 30 Azide blood agar test chamber 52 reinforcing the gram-positive nature of those microorganisms.

The next five examples (FIG. 31-FIG. 35) are all gram-negative microorganisms. See the table immediately below for a description of the figures. These figures will be discussed as a group. Note the absence of growth in all Azide blood agar test chambers 52 and the growth in various MAC media (chambers 53, chambers 54, chambers 55, chambers 57, chambers 58, chambers 59, and chambers 65), indicating that all are gram-negative. MUG MAC agar test chambers 65 show growth and fermentation of the lactose containing media in FIG. 31 and FIG. 32 and no-growth in FIG. 33-FIG. 35. More importantly, FIG. 31, (the E coli inoculated kit plate), contains the only MUG-MAC agar chamber 65 that fluoresces when irradiated with UV light at 365 nm (see FIG. 39E for example). FIG. 33, which is the Pseudomonas aeruginosa inoculated kit plate, contains the only pseudomonas F agar test chamber 63 where green pigment is produced (fluorescein). Additionally, irradiation of fluorescein by UV light at 365 nm produces fluorescence (not shown). Note the Hektoen agar test chambers 60 showing growth in FIG. 32, and color change and growth in FIG. 33 and FIG. 35 characteristic of Pseudomonas aeruginosa and Salmonella species. Note the production of Pyocyanin pigment in the Mueller Hinton test chambers (67′-70′ and 72′-75′), in FIG. 33 indicative of Pseudomonas aeruginosa.

The preferred embodiment kit plate contains six test chambers (53, 54, 55, 57, 58, and 59) designed to measure the ability of the test organism to ferment a particular carbohydrate. The carbohydrates used are respectively Lactose, Glucose, Mannitol, Inositol, Sucrose and Arabinose. In addition, MacConkey agar-based media used in the kit plate is selective for the growth of gram-negative organisms only. When fermentation takes place, the medium becomes acidified resulting in red to pink colonies of the bacteria. For example, fermentation is obvious in FIG. 31 test chambers 53, 54, 55, and 59. Contrast this result to FIG. 31 test chambers 57 and 58 where there is growth but no fermentation. The table below lists the results of citrate utilization and fermentation for FIG. 31 through FIG. 35 where “1” denotes a positive result and “0” denotes a negative result. Compare to FIGS. 24A-24C

| Test chamber number | 62 | 59 | 54 | 57 | 53 | 55 | 58 |
|---|---|---|---|---|---|---|---|
| Gram-negative organism | CIT | ABA | GLU | INO | LAC | MAN | SUC |
| E. coli | 0 | 1 | 1 | 0 | 1 | 1 | 0 |
| Klebsiella pneumoniae | 1 | 1 | 1 | 1 | 1 | 1 | 1 |
| Pseudomonas aeruginosa | 1 | 0 | 1 | 0 | 0 | 0 | 0 |
| Proteus vulgaris | 0 | 0 | 1 | 0 | 0 | 0 | 1 |
| Salmonella typhimurium | 1 | 1 | 1 | 0 | 0 | 1 | 0 |

FIG. 36 shows an example of the preferred embodiment kit plate incubated for 48 hrs after inoculation with the fungus Candida albicans ATCC 14053. Perhaps most obvious is the lack of inhibition by any of the eight antimicrobial agents. None of these agents is active against fungus. Note the growth of colonies in Littman oxgall agar 71. This is a useful medium for the isolation of fungi and suitable for the growth of pathogenic fungi. Molds and yeasts form non-spreading, discrete colonies as seen with the Candida here.

FIGS. 37A to 37C illustrates the growth of two microorganisms, Staph. aureus and E. coli, together on the same preferred embodiment kit plate. Both organisms, from stock culture, inoculate the BHIB 82. Following 4 hr incubation at 35° C., two different dilutions are made from the IIBHIB 90 and inoculated on the kit plate as described previously. See above for a complete description of the method. To serve as an example of how to use this kit and method for ID testing and antimicrobial testing of an unknown when more than one microorganism is present, assume that it is unknown what the microorganism or microorganisms are in FIG. 37A. The first step is to take a small volume from the IIBHIB 90, place it on a glass slide and observe the sample under a 400× to 600×microscope. What type of cellular morphology is present? Are there multiple forms? Is there motility? Motile rods and cocci in clusters are present. A sample from the IIBHIB 90 is gram stained. The second step is to look at the colonies on the kit plate in the different test chambers. A useful magnifier is 10× microscope eyepiece turned upside down. How many different types of colonies are there? There appears to be two types on the blood agar 51. One of the colonies is hemolytic. Refer to FIG. 10 for a review of the different colony characteristics.

If there are more than two types of microorganisms on the kit plate, consider the following: Although polymicrobic infections do occur, particularly when mixed bacterial species are recovered from deep wounds or visceral organs, this same mixture of organisms from culture of urine, the respiratory tract, or superficial skin wounds or ulcers must be interpreted differently. R. C. Bartlett (Am. J. Clinical. Pathology 61: 867-872, 1974) has recommended that routine cultures that grow three or more organism types should not be further processed. The recovery of three or more organisms from specimens obtained from non-sterile sites most commonly represent colonization or contamination. Repeat cultures may be indicated of there is clinical evidence of infection. Others have reported similar experiences to that reported by Bartlett: that repeat cultures rarely confirm isolation of the same bacterial pathogens.

The third step in the ID of the hypothetical unknown in FIG. 37A is to perform a cytochrome oxidase assays on the colonies growing on the MacConkey media. The results, from testing several colonies from the MacConkey test chambers, are negative for cytochrome oxidase. A nitrate reductase test on the IIBHIB 90 show that nitrate is reduced during the incubation.

The forth step involves observing the test chambers. FIG. 37A reveals organisms growing on the azide blood agar 52 as well as on a number of the Mac test chambers (53, 54, 55, 57, 58, 59, 65). This implies a gram negative and gram-positive organism on the kit plate. Neither organism utilizes citrate as seen by the non-reacted green medium in citrate test chamber 62. Mannitol salt agar 61 exhibits growth and is acidic (note yellow color of agar) indicating Staphylococcus. In addition, the Tellurite Glycine agar 66 also exhibits growth and is black, which identifies the Staph. as coagulase positive. One strong possibility is Staph. aureus.

In step 5, it is found that the MacConkey media shows only one type of colony. Since the organism is growing on the MacConkey media, it is gram negative (also recall the above gram stain results). The organism ferments the following sugars in the respective test chambers: Lactose 53, glucose 54, Mannitol 155, and arabinose 59. The organism does not ferment inositol 57 or sucrose 58. Step 6 takes the information obtained to this point, and applies the accumulated criteria to the database of FIGS. 24A-24C. The data are filtered to extract out the possible microorganisms with this set of criteria. Following is a copy of the results of that extraction:

| GRAM NEGATIVE | | | | | | | | | | | | |
|---|---|---|---|---|---|---|---|---|---|---|---|---|
| ORGANISM | INCUBATION | CIT | ARA | GLU | INO | LAC | MAN | SUC | OXI | NO2 | MOT | MAC |
| E. coli | 12-20 h | 0 | 1 | 1 | 0 | 1 | 1 | 0 | 0 | 1 | 1 | 1 |
| Escherichia fergusonii | 12-20 h | 0 | 1 | 1 | 0 | 1 | 1 | 0 | 0 | 1 | 1 | 1 |
| Escherichia vulneris | 12-20 h | 0 | 1 | 1 | 0 | 1 | 1 | 0 | 0 | 1 | 1 | 1 |

E. coli is the most common gram-negative microbe isolated and identified in clinical microbiology laboratories. Methylumbelliferyl-beta-D-glucuronide (MUG) is a substrate of the E. coli enzyme beta-glucuronidase. MUG becomes fluorescent when this enzyme cleaves it. Incorporating MUG directly into a modified MacConkey agar allows for the direct detection of E. Coli. (J. Clinical. Microbiology 1984 Feb; 19(2): 172-4). The preferred embodiment kit plate incorporates this medium (MUG-MAC test chamber 65. Step 7 involves irradiating test chamber 65 with UV light at 365 nm. Fluorescence is observed in FIG. 37A, MUG-MAC test chamber 65 (not specifically illustrated but see FIG. 39E for example). The fluorescence confirms E. coli as the other microorganism present

The AST portion of the kit plate shown in FIG. 37A involves the following test chambers: Ampicillin test chamber 67′, Augmentin test chamber 68′, Amikacin test chamber 69′, Cephalothin test chamber 70′, Doxycycline test chamber 72′, Enrofloxicin test chamber 73′, Gentamicin test chamber 74′, and Septra test chamber 75′. Since there are two microorganisms on the above kit plate, they will both be present in the AST chambers. In the event that both organisms are pathogens, the choice would be to treat with the antimicrobial agent which both organisms are sensitive too. Take measurement from the antimicrobial corners to the margins for each microorganism. Then compare these values to the values in the MODIFIED INTERPRETIVE STANDARDS TABLE ([0071]). This allows one to determine if the organism is Sensitive, Intermediate or Resistant for each of the antimicrobial agents.

To determine which margin belongs to which microorganism the following will be helpful. For ease of viewing the margins of each microorganism, FIGS. 37B and 37C provide two different enlarged views of the FIG. 37A AST chambers (67′-70′ and 72′-75′). FIG. 37B is a view with back lighting. FIG. 37C is a view with front lighting. Two AST chambers featured in these figures are the Mueller Hinton agar plus antimicrobial Cephalothin test chamber 70 ′ and the Mueller Hinton agar plus antimicrobial Enrofloxicin test chamber 73′. Two margins are observable in each test chamber: E coli margin 180 and Staph. aureus margin 181 for the Cephalothin test chamber 70′, and Staph. aureus margin 182 and E. coli margin 183 for the Enrofloxicin test chamber 73′. The region 176A is found to consist of E. coli and the region 178A is found to consist of Staph. aureus as confirmed by gram staining (FIG. 38) both regions. 176A=176B(gram stained negative rod)=E. coli and 178A=178B(gram stained positive cocci)=Staph. aureus. Where the cellular morphology is different enough, it would be sufficient to do wet mounts of the regions between the margins to ascertain specific antimicrobial susceptibilities. There is value in doing AST on the mixture of organisms found in an infectious site. Suppose a hypothetical organism-A possesses an enzyme that inactivates the penicillin antimicrobials. The other organism-B however is sensitive to the penicillins. If the susceptibilities had been done separately or only on organism-B perhaps penicillin would have been used to treat the infection. However, since organism-A is also present, it would inactivate the antimicrobial therapy. The fact is that in the above mixture of Staph aureus and E. coli, the Staph. does indeed possess the penicillin destroying B-lactamase enzyme.

The last example again involves a mixture of two organisms. However, this time they are both gram negative and both grow on MacConkey media. FIG. 39A is an example of a preferred embodiment kit plate inoculated with a mixture of two microorganisms, E. coli and Salmonella typhimurium. The sets of figures illustrate the utility of the method and kit when several gram-negative microorganisms are present. FIG. 39A shows the preferred embodiment kit plate and the result of growth and biochemistry of the organisms on the various media. The antimicrobial susceptibilities appear to be quite similar. There is no growth in the Azide test chamber 52, which illustrates gram-negative organisms only. Citrate is utilized but it may not clear which organism is utilizing it. FIG. 39A Hektoen test chamber 60 indicates Salmonella is one of the microorganisms. A closer view, a 10× magnification of test chamber 65 shows the black centered salmonella colonies. A close 10×view of the blood agar test chamber 51 reveals two different types of colonies (FIG. 39B) based on size: An E. coli colony 184 and a Salmonella typhimurium colony 186. FIG. 39E illustrates fluorescence in the MUG MacConkey test chamber 65, due to the action of E. coli beta-glucuronidase enzyme on the substrate 4-methylumbelliferyl-beta-D-glucuronide. E. coli is shown to be the other gram-negative. A further look at test chambers glucose 54, mannitol 55, and arabinose 59 show both organisms fermenting those sugars. In addition, inositol 57 and sucrose 58 test chamber show both organisms non-fermenting the sugar substrates. So far, the results match the published criteria. Both organisms ferment glucose, mannitol and arabinose and do not ferment inositol and sucrose. The difference lies in how they handle Lactose. E. coli ferments it but Salmonella typhimurium does not. A 20× view of the MacConkey lactose test chamber 53, FIG. 39D supports the ID's. E. coli colony 188 is fermenting lactose whereas Salmonella typhimurium colony 190 is not fermenting lactose.

### Additional Embodiments

The method and kit is adaptable for the ID and AST of a broad number of microorganisms comprising gram-positive bacteria, gram-negative bacteria, higher bacteria and Mycoplasma, and fungi. The choice of broth used for the initial inoculation may be selected from a number of media that support the growth of the specific type of microorganism in question. In addition, the specimen may be inoculated into any number of growth media and not necessarily a broth type medium. In certain circumstances, such as when a particular organism is suspect, the broth may be rendered selective at the onset with the addition of any number of agents. For example, a specimen possibly containing the gram-positive Bacillus anthracis (Anthrax) needs ID and AST. Let the broth be selective such as Brain-Heart infusion broth plus 50 units/ml of Polymyxin B. This initial incubation media will inhibit most of the gram-negative microorganisms that could be present in a specimen, and favor growth of gram-positive microorganisms. Within 4 to 8 hrs, there will be a sufficient number of microorganisms to inoculate the kit plate. As another embodiment, the multi-chambered kit plate media can include several selective and differential media useful for Bacillus: Bacillus cereus selective agar (BCA) and/or Phenylethanol agar with 5% defibrinated sheep blood. Anthrax (Bacillus anthracis) is a large spore-forming gram-positive rod (1-1.5×3-10 micron) that forms oval, central to sub-terminal spores (1×1.5 micron) that do not cause swelling of the cell. It grows in culture as gray-white colonies, generally flat or slightly convex with characteristic comma-shaped protrusions. The edges are slightly undulate and have a ground glass appearance. Anthrax is differentiated from other gram-positive rods on culture by lack of hemolysis, lack of motility and by preferential lack of growth on Phenylethyl alcohol blood agar. Other Bacilli are generally hemolytic, motile and grow on Phenylethyl alcohol blood agar.

FIG. 1 illustrates one of the kit components, a multi-chambered polystyrene plate having 25 square test chambers and ethylene oxide sterilized. In other embodiments, it is possible for the kit plate to have test chambers of any dimension and any composition of plastic material such as polypropylene where the plastic can be formed into a multi-chambered kit plate that can be sterilized. In the case of polypropylene, the kit plate can be steam sterilized instead of ethylene oxide sterilized. Other numbers of test chambers per kit plate can be produced and utilized. In another embodiment, the kit plate can contain circular or rectangular test chambers or any shape of chamber with sufficient surface for observing bacterial growth.

Microorganism pathogens of animals are frequently classified into two groups: extra cellular and facultative intracellular bacteria and the obligate intracellular and cell associated bacteria. Over 50 genera of extra cellular and facultative intracellular bacteria are listed in the first following table ([0092]) with their staining reactions, cellular characteristics, and oxygen requirements. Currently all of the non-fastidious microorganisms (marked with “−” under “Fastidious growth requirements”) will grow on the preferred embodiment kit plate. Those that have fastidious growth requirements and/or are anaerobic in their requirement for oxygen will not grow on the preferred embodiment kit plate. Additional embodiments with different media and/or different gas environments will allow for growth and characterization of these fastidious organisms. The second table ([0093]) lists additional usable media in place of the preferred embodiment media. Other embodiments would comprise different combinations of the medium listed below as well as newly developed formulations. Mueller Hinton medium, used in the preferred embodiment, may be enriched with other nutrients in another embodiment. Any other suitable AST medium can be used that will allow for reliable AST. In another embodiment, an AST media can be utilized that comprises a selective agent to eliminate unimportant microorganisms, allowing only for the AST of particular pathogens.

An embodiment where anaerobic microorganisms are AST tested, would utilize a set of antimicrobial agents with clinical indications against anaerobic bacteria. Examples are Clindamycin, Imipenem, Ampicillin-Sublactam, and Metronidazole. It is important to note, concerning anaerobes, that resistance among the B. fragilis group is increasing, while certain Clostridia species are frankly resistant, and therefore AST of anaerobes is very desirable. In another embodiment, additional wells or test chambers are utilized for AST with any available antimicrobial agent under any atmosphere.

In addition to the use of different media, is the option of culturing in different gas atmospheres. These other gas environments are possible with commercial systems. Anaerobic incubators of any brand and make will suffice. A convenient alternative is the pouch systems for the anaerobic incubation of up to two of the preferred embodiment kit plates. These systems comprise a plastic see-through pouch and a paper gas-generating sachet. The paper sachet contains ascorbic acid and activated carbon that react on contact with air. Oxygen is rapidly absorbed and carbon dioxide produced. When the paper sachet is placed in a sealed plastic pouch, the reaction creates ideal atmospheric conditions for the growth of anaerobes.

TABLE
CHARACTERISTICS OF GENERA OF EXTRA CELLULAR AND FACULTATIVE
INTRACELLULAR BACTERIA

| | | | | O2 | |
|---|---|---|---|---|---|
| Genus | Cell shape | Gram stain | Motility | requirement | Fastidious growth requirements |
| Micrococcus | Cocci | + | − | Aerobic | − |
| Staphylococcus | Cocci | + | − | Aerobic | − |
| Streptococcus | Cocci | + | − | Aerobic | − |
| Bacillus (has endospores) | Rods | + | − | Aerobic | − |
| Corynebacterium | Rods | + | − | Aerobic | − |
| Dermatophilus | Rods | + | + | Aerobic | − |
| Erysipelothrix | Rods | + | − | Aerobic | − |
| Listeria | Rods | + | + | Aerobic | − |
| Nocardia | Rods | + | − | Aerobic | − |
| Propionibacterium | Rods | + | − | Aerobic | − |
| Rhodococcus | Rods | + | − | Aerobic | − |
| Acinetobacter | Rods | − | − | Aerobic | − |
| Actinobacillus | Rods | − | − | Aerobic | − |
| Aeromonas | Rods | − | + | Aerobic | − |
| Alcaligenes | Rods | − | + | Aerobic | − |
| Bordetella | Rods | − | + | Aerobic | − |
| Citrobacter | Rods | − | + | Aerobic | − |
| Edwardsiella | Rods | − | + | Aerobic | − |
| Enterobacter | Rods | − | + | Aerobic | − |
| Escherichia | Rods | − | + | Aerobic | − |
| Klebsiella | Rods | − | − | Aerobic | − |
| Moraxella | Rods | − | − | Aerobic | − |
| Morganella | Rods | − | + | Aerobic | − |
| Pasteurella | Rods | − | − | Aerobic | − |
| Proteus | Rods | − | + | Aerobic | − |
| Providencia | Rods | − | + | Aerobic | − |
| Pseudomonas | Rods | − | + | Aerobic | − |
| Salmonella | Rods | − | + | Aerobic | − |
| Serratia | Rods | − | + | Aerobic | − |
| Shigella | Rods | − | − | Aerobic | − |
| Vibrio | Rods | − | + | Aerobic | − |
| Yersinia | Rods | − | + | Aerobic | − |
| Neisseria | Cocci | − | − | Aerobic | + |
| Mycobacterium | Rods | + | − | Aerobic | + |
| Borrelia | Rods | − | + | Aerobic | + |
| Brucella | Rods | − | − | Aerobic | + |
| Campylobacter | Rods | − | + | Aerobic | + |
| Francisella | Rods | − | − | Aerobic | + |
| Haemophilus | Rods | − | − | Aerobic | + |
| Legionella | Rods | − | + | Aerobic | + |
| Leptospira | Rods | − | + | Aerobic | + |
| Mycoplasma | Rods | − | − | Aerobic | + |
| Taylorella | Rods | − | − | Aerobic | + |
| Peptococcus | Cocci | + | − | Anaerobic | + |
| Peptostreptococcus | Cocci | + | − | Anaerobic | + |
| Veillonella | Cocci | − | − | Anaerobic | + |
| Antinomies | Rods | + | − | Anaerobic | + |
| Bifidobacterium | Rods | + | − | Anaerobic | + |
| Clostridium (has endospores) | Rods | + | + | Anaerobic | + |
| Eubacterium | Rods | + | − | Anaerobic | + |
| Bacteroides | Rods | − | − | Anaerobic | + |
| Fusobacterium | Rods | − | − | Anaerobic | + |
| Treponema | Rods | − | + | Anaerobic | + |

TABLE OF MEDIA AND THEIR USEFULNESS

| Medium | Usefulness |
|---|---|
| A8 agar | Isolating and differentiating genital strains of mycoplasmas. |
| Actinomycete Isolation Agar | Isolating Actinomycete from soil and water |
| Agar Medium No. F | Detecting Enterobacteriaceae and other gram-negative bacteria in pharmaceutical products |
| American trudeau Society medium | Cultivation of acid-fast bacteria (mycobacteria) |
| Anaerobic Agar | Cultivating anaerobic microorganisms |
| Azide Blood Agar Base | Isolating streptococci and staphylococci; for use with blood in determining hemolytic reactions |
| Bacillus cereus selective agar (BCA) | Isolating and differentiating Bacillus anthracis in meat and tissue |
| Bacteroides Bile-Esculin agar | Isolation and ID of Bacteroides fragilis group and Biophilia spp. |
| BG Sulfa Agar | Isolating salmonella |
| Baird-Parker Agar Base | Isolating and enumerating staphylococci in foods and other materials |
| BIGGY Agar | Isolating and differentiating Candida spp. |
| Bile Esculin Agar Base | Isolating and presumptively identifying group D streptococci |
| Bile Esculin Agar | Isolating and presumptively identifying group D streptococci and Enterococcus spp. |
| Bile Esculin Azide Agar | Isolating, differentiating and presumptively identifying group D streptococci |
| Bismuth sulfite agar | Selective for Salmonella spp. |
| Blood agar, anaerobic (CDC) | General growth medium for anaerobic bacteria |
| Blood agar, anaer. W K & Val | Isolation of Bacteroides spp. And other obligately anaerobic bacteria |
| (CDC) | |
| Blood Agar Base | Isolating and cultivating a wide variety of microorganisms. Plus blood, fastidious organisms |
| Blood Agar Base No. 2 | Isolating and cultivating fastidious microorganisms with or without added blood |
| Blood Agar, Laked, anaerobic with | Isolation of Bacteroides spp. And other obligately anaerobic bacteria with enhanced Prevotella pigment production |
| K & VA | |
| Blood Agar, Phenylethyl alcohol, | Isolation of Bacteroides spp., Prevotella spp., and other obligately anaerobic bacteria from facultative anaerobes. |
| anaerobic | |
| Bordet Gengou Agar Base | Isolating Bordetella pertussis and other Bordetella species |
| Brain Heart Infusion Agar | Cultivating fastidious microorganisms, especially fungi and yeasts |
| Brain Heart CC Agar | Isolating and cultivating fastidious fungi |
| Brain Heart Infusion w/PAB and | Cultivating fastidious organisms, particularly from blood containing sulfonamides |
| Agar | |
| Brewer Anaerobic Agar | Cultivating anaerobic and microaerophilic bacteria |
| Brilliant Green Agar | Isolating Salmonella other than Salmonella typhi |
| Brilliant Green Agar Modified | Isolating Salmonella from water, sewage and foodstuffs |
| Brilliant Green Bile Agar | Isolating, differentiating and enumerating coliform bacteria |
| Brucella Agar | Isolating and cultivating Brucella |
| Campylobacter Agar Base | Isolating and cultivating Campylobacter |
| Candida BCG Agar Base | Isolating and differentiating Candida from primary specimens |
| Candida Isolation Agar | Isolating and differentiating Candida albicans |
| Cetrimide Agar Base | Isolating and cultivating Pseudomonas aeruginosa |
| Charcoal Agar | Cultivating fastidious organisms, especially Bordetella pertussis fro vaccine production |
| Chocolate Agar | Supports growth of Neisseria and Haemophilus |
| Clostridium difficile selective media | Isolating C. difficile from fecal specimens of patients |
| Columbia Blood Agar Base EH | Isolating and cultivating fastidious microorganisms when used with blood |
| Columbia Blood Agar Base | Cultivating fastidious microorganisms with or without the addition of blood |
| Columbia Blood Agar Base No. 2 | Isolating and cultivating fastidious microorganisms when used with blood |
| Cooke Rose Bengal Agar | Isolating fungi from environmental and food specimens |
| Corn Meal Agar | Stimulating the production of chlamydospores by Candida albicans |
| Cystine Heart Agar | Cultivating Francisella tularensis when used with blood |
| Cystine Tryptic Agar | Used with added carbohydrates in differentiating microorganisms based on fermentation reactions |
| Czapek Solution Agar | Cultivating fungi and bacteria capable of using inorganic nitrogen |
| DCLS Agar | Isolating gram-negative enteric bacilli |
| D/E Neutralizing Agar | Used for neutralizing and determining the bactericidal activity of antiseptics and disinfectants |
| DNase Test Agar w/Methyl Green | Identify potentially pathogenic staphylococci based on deoxyribonuclease activity |
| DRBC Agar | Enumeration of yeasts and molds |
| Desoxycholate Agar | Isolating and differentiating gram-negative enteric bacilli |
| Desoxycholate Citrate Agar | Isolating enteric bacilli, particularly Salmonella and many Shigella species |
| Desoxycholate Lactose Agar | Isolating and differentiating gram-negative enteric bacilli and enumerating coliforms from water, wastewater, |
| | diary |
| Dextrose Agar | Cultivating a wide variety of microorganisms with or without added blood |
| Dextrose Starch Agar | Cultivating pure cultures of Neisseria gonorrhoeae and other fastidious microorganisms |
| Dextrose Tryptone Agar | Cultivating thermophilic “flat-sour” microorganisms associated with food spoilage |
| Differential Reinforced Clostridial | Cultivating and enumerating sulfite-reducing clostridia |
| Agar | |
| Dubos Oleic Agar Base | Isolating and determining the susceptibility of Mycobacterium tuberculosis |
| Egg Yolk Agar | Differentiate species of anaerobic and aerobic bacteria based on detection of lecithinase, lipase, and |
| | protease activity |
| M E Agar | Isolating and differentiating enterococci from water by membrane filtration |
| Esculin Iron Agar | Enumerating enterococci from water by membrane filtration based on esculin hydrolysis |
| EMB Agar | Isolating and differentiating gram-negative enteric bacilli |
| Emerson YpSs Agar | Cultivating Allomyces and other fungi |
| Endo Agar | Confirming the presence of coliform organisms |
| M Enterococcus Agar | Isolating and enumerating enterococci in water and other materials by membrane or pour plate techniques |
| Eugon Agar | Cultivating a wide variety of microorganisms, particularly in mass cultivation procedures. |
| M FC Agar | Cultivating and enumerating fecal coliforms by membrane filter technique at elevated temperatures |
| HC Agar Base | Enumerating molds in cosmetic products |
| M HPC Agar | Enumerating heterotrophic organisms in treated potable water and other water samples by membrane filtration |
| Heart Infusion Agar | Cultivating a wide variety of fastidious microorganisms and as a base for preparing blood agar |
| Hektoen Enteric Agar | Isolating and differentiating gram-negative enteric bacilli |
| KE Streptococcus Agar | Isolating and enumerating fecal streptococci according to APHA |
| LPM Agar Base | Isolating and cultivating Listeria monocytogenes |
| Lactobacilli MRS Agar | Isolation, enumeration and cultivation of Lactobacillus species |
| Letheen Agar | Evaluating the bactericidal activity of quaternary ammonium compounds |
| Lima Bean Agar | Cultivating fungi |
| Lift man Oxgall Agar | Isolating and cultivating fungi, especially dermatophytes |
| Liver Infusion Agar | Cultivating Brucella and other pathogenic organisms |
| Liver Veal Agar | Cultivating anaerobic microorganisms |
| M 17 Agar | Enumerating lactic streptococci in yogurt, cheese starters and other dairy products |
| MYP Agar | Enumerating Bacillus cereus from foods |
| MacConkey Agar | isolating and differentiating lactose fermenting from non-fermenting gram-negative enteric bacilli |
| MacConkey Agar Base | Used with added carbohydrates in differentiating microorganisms based on fermentation reactions |
| MacConkey Agar CS | Isolating and differentiating gram-negative enteric bacilli from specimens containing swarming strains of proteus |
| MacConkey Agar w/o Salt | Isolating and differentiating gram-negative bacilli while suppressing the swarming of most proteus species |
| MacConkey Agar w/o CV | Isolating and differentiating enteric microorganisms while permitting growth of staphylococci and enterococci |
| MacConkey Sorbitol Agar | Isolating and differentiating enteropathogenic E. coli serotypes |
| Malt Agar | Isolating and cultivating yeasts and molds from food, and for cultivating yeast and mold stock cultures |
| Malt Extract Agar | Isolating, cultivating and enumerating yeasts and molds |
| Mannitol Salt Agar | Isolating and differentiating staphylococci |
| McBride Listeria Agar | Isolating Listeria monocytogenes with or without the addition of blood |
| McClung Toabe Agar Base | Isolating and detecting Clostridium perfringens in foods based on the lecithinase reaction |
| Microbial Content Test Agar | Detection of microorganisms on surfaces sanitized with quaternary ammonium compounds |
| Mueller-Hinton medium plain | Testing bacteria for susceptibility to antimicrobial agents |
| Mueller-Hinton m. with 5% sheep B. | As above with MH plain plus testing strains of Streptococcus spp. And other fastidious bacteria |
| Mueller-Hinton m. chocolatized | As above for MH plain, MH with 5% sheep blood plus testing Haemophilus and Neisseria |
| Mycobacteria 7HI I Agar | Isolating, cultivating and AST testing of fastidious strains of mycobacteria |
| Milk Agar | Enumeration of microorganisms in liquid milk, ice cream, dried milk and whey |
| Mitis Salivarius Agar | Isolating Streptococcus mitis, S. salivrius and enterococci, particularly from grossly contaminated specimens |
| Modified Letheen Agar | Microbiological testing of cosmetics |
| Mycobiotic Agar | Isolating pathogenic fungi |
| Mycological Agar | Cultivating fungi at a neutral pH |
| Mycological Agar w/Low pH | Isolating and cultivating fungi and aciduric bacteria |
| Oatmeal Agar | Cultivating fungi, particularly for macrospore formation |
| Orange Serum Agar | Cultivating aciduric microorganisms, particularly those associated with spoilage of citrus products |
| PPLO Agar | Isolating and cultivating Mycoplasma |
| Peptone Iron Agar | Detecting hydrogen sulfide production by microorganisms |
| Phenylethanol Agar | Isolating gram-positive microorganisms but markedly to completely inhibiting gram-negative microorganisms |
| Phenylalanine Agar | Differentiating Proteus and Providencia species from other Enterobacteriaceae based on defamations of |
| | phenylalanine |
| Potato Dextrose Agar | Culturing yeasts and molds from food and dairy products |
| Protease No. 3 Agar | Isolating and cultivating Neisseria and Haemophilus |
| Pseudomonas Agar F | Detecting the production of fluorescein. Produced by P. seruginosa, P. putida, P. fluroescens and unidentified |
| | fluor. P. |
| Pseudomonas Agar P | Detecting and differentiating Pseudomonas aeruginosa from other pseudomonas based on pyocyanin preduction |
| Pseudomonas Isolation Agar | Isolating Pseudomonas and differentiating Pseudomonas aeruginosa from other pseudomonads based on pigment |
| Rice Extract Agar | Differentiating Candida albicans and other Candida spp. Based on chlamydospore formation |
| Rose Bengal Agar Base | Isolating and enumerating yeasts and molds |
| SABHI Agar Base | Isolating and cultivating pathogenic fungi |
| SPS Agar | Detecting and enumerating Clostridium perfringens in food |
| Sabouraud Dextrose Agar | Culturing yeasts, molds and aciduric microorganisms |
| Salmonella-Shigella Agar | Isolation of Salmonella spp. And many strains of Shigella spp. From feces |
| Sabouraud Maltose Agar | Culturing yeasts, molds and aciduric microorganisms |
| Simmons Citrate Agar | Differentiation of enteric gram-negative bacilli from clinical specimens, water samples, and food samples |
| Spirit Blue Agar | Detecting and enumerating lipolytic microorganisms in diary products |
| TCBS Agar | Isolating and cultivating Vibrio cholerae and other enteropathogenic vibrios |
| M TEC Agar | Isolating, differentiating and enumerating thermotolerant E. coli from water by membrane filtration |
| TPEY Agar Base | Detecting and enumerating coagulase-positive staphylococci |
| Tellurite Glycine Agar | Isolating coagulase-positive staphylococci |
| Thermoacidurans Agar | Isolating and cultivating Bacillus coagulans (Bacillus theremoacidurans) from foods |
| Thiosulfate citrate bile salts sucrose | Isolating Vibrio cholerae and other pathogenic vibrios from samples of feces and food containing mixed species |
| agar | |
| Tomato Juice Agar | Cultivating and enumerating Lactobacillus species |
| Tomato Juice Agar Special | Cultivating and enumerating lactobacilli and other acidophilic microorganisms from saliva and other specimens |
| Triple Sugar Iron Agar | Differentiating gram-negative enteric bacilli based on fermentation of dextrose, lactose, sucrose and on H25 |
| | production |
| Tryptic Soy Agar | Isolating and cultivating fastidious microorganisms and, with blood, in determining hemolytic reactions |
| Tryptone Glucose Extract Agar | Cultivating and enumerating microorganisms in water and dairy products |
| Tryptose Agar | Isolation of Brucella from blood |
| Tryptose Blood Agar Base | Isolating, cultivating and determining the hemolytic reactions of fastidious microorganisms |
| VJ Agar | Isolating coagulase-positive, mannitol-fermenting staphylococci |
| Veal Infusion Agar | Cultivating fastidious microorganisms with or without the addition of blood |
| Veillonella Agar | Isolating Veillonella when used with vancomycin |
| Violet Red Bile Agar | Enumerating coliform organisms in dairy products |
| Violet Red Bile Agar with MUG | Enumerating E. coli and total coliform bacteria in food and dairy products |
| Violet Red Bile Glucose Agar | Detecting and enumerating Enterobacteriaceae in food and dairy products |
| XLD Agar | Isolating and differentiating gram-negative enteric bacilli, especially Shigella and Providencia |
| XLT4 Agar Base | Isolating non-typhi Salmonella |
| YM Agar | Cultivating yeasts, molds and other aciduric microorganisms |
| Yeast Extract Glucose | Enumerical yeasts and molds in milk and milk products (recommended by International dairy Federation) |
| Chloramphenicol Agar | |
| Yersinia Selective Agar Base | Isolating and cultivating Yersinia enterocolitica |

### Conclusions, Ramifications, and Scope

Thus, the reader will see that the method and kit described above in this patent application provides a novel and unique diagnostic tool for the characterization of unknown microorganisms from any source. The advantages take on significant meaning in a world where the unseen microscopic enemy either conquers or is conquered. The outcome depends on the readiness of the body's defense system to fight the pathogen plus how quickly the organism is identified, susceptibility tested and treatment started. The sooner the administration of the right antibiotic, the better the chance is for winning the battle. Listed below are several advantages of using this kit and method.

The results (concurrent ID and AST) are obtainable in one-third the time of standard methods, usually within 24 hrs. This is a critical advantage in situations of life-threatening illnesses where it is important to know which antibiotic to use as well as the ID of the pathogen.

The kit is cost effective and complete with no additional items needed.

The specimen is directly inoculated into the kit broth with no delay in transporting the specimen.

Generally within 4 to 6 hrs, the broth culture is diluted and inoculated onto the ID-AST kit plate. The antimicrobial portion (AST) shows visible results even by 8 hrs, with the faster growing Enterobacteriaceae family of microorganisms

The kit can be used anywhere that an incubation temperature can be maintained (35° C.-37° C.).

The kit is versatile in that many different types of organisms are tested at the same time.

Since there is no initial isolation step, there is little likelihood of errors in judgment.

The AST portion of the kit is also novel and unique in that the end-of-incubation measurements correlate exactly (x½) to the standard Kirby-Bauer disk-diffusion AST system. Any set of antimicrobial agents can be tested and more than one microorganism can exist in the same test chamber and still be analyzed (see above).

A paradigm in microbiology is that isolated colonies are required (i.e. “pure cultures”) before any identification testing can begin. Streak plates are prepared and incubated for that purpose. Eighteen to 24 hrs later, the colonies that form are tested by picking them from the plate and transferring for additional growth (18-24 hrs) in identification systems or ID media. When the ID is established, an additional 18-24 hrs are required to do AST for each microorganism deemed important. Another paradigm states that to do an AST test it is again required to first isolate the organism(s) of interest. The present method and kit allows for a significant short cut with no sacrifice to reliability. Isolations and identifications of several microorganism types take place together in the same chambers at the same time without the need for an initial 18 to 24 hrs isolation step first. Broth is inoculated instead, taking generally 4 to 6 hrs to grow up the microorganisms. Then dilutions are made and inoculated into the kit plate that performs the testing (ID concurrent with AST) in normally 12 to 20 hrs. In certain cases, selective or single purpose media will perform the “isolating”, because only one type of organism will grow on a particular medium. Two examples are the ID of Enterococcus on Bile Esculin Azide agar or the ID of Coagulase-positive staphylococcus on Tellurite Glycine agar. Reliable ID and AST, using the novel kit and method, does take place directly from broth culture.

While my above description contains many specificities, these should not be construed as limitations on the scope of the invention, but rather as an exemplification of one preferred embodiment thereof. Many other variations are possible. For example, the inclusion of Iota carrageenan into the media provides a stabilization of the agar-based media and therefore increases the shelf life of the kit. Iota carrageenan in conjunction the agar in the media at several different ratios, results in a stronger gel, elastic and cohesive with little syneresis (watering out). In addition, the gel is more stable to freeze-thaw conditions. While I believe this information is correct due to studies performed by me, I do not wish to be bound by this.

In order for the claims to be interpreted as broadly as possible, listed below are some variations of a number of the elements of the present kit and method. The inoculation of the initial broth culture can be done by using any number of different elements besides a swab. For instance, a syringe and needle serves this purpose as well as any other device that will sample the point of interest containing the microorganism for study. The type of incubation vessel can be any number of different materials. The culture atmosphere can comprise any type and mixture of gas. The way of determining and preparing the density of the bacterial growth for study can be by any number of methods from the McFarland standards to a spectrophotometric determination. The method of inoculating the multi-chambered kit plate can also be different than in the preferred embodiment. From a multi-pipette to spraying on the inoculum would be appropriate. Any that will allow for the even distribution of inoculum is permissible. Other chemistries that would elucidate the identification of an unknown microorganism from the unique colony of the organism such as newer methods of molecular biology would be permissible such as PCR, immunological methods or other heretofore undiscovered to assay the composition of the cellular DNA, antigenic nature, or other molecular features of the specific microorganism.

The process of applying the antimicrobial agents on the kit plate can be done with other devices than the one shown in the preferred embodiment such as tweezers, forceps, vacuum devices, static electricity, air driven applicators or any other of placement. The preferred embodiment disk quarter is unique in the shape of the antimicrobial agent carrier in terms of the equivalence to standard methods. A prior art reference cited the use of antimicrobial disks at one end of elongated channel containing plates (U.S. Pat. No. 6,251,624). This patent listed embodiments with different sizes of disks but not different shapes in contrast to the disk quarter of the current preferred embodiment. In addition, it would be possible to expand the geometry with a “disk-half” for setting at the midpoint of an edge of a test chamber that would provide similar equivalence. It would however be less economical by one-half.

Included in the kit are reagents and analytical papers for the determination of nitrate reductase and cytochrome oxidase activity in the microorganisms growing from the specimen. However, other reagents in various forms can be utilized in the method. Other embodiment could utilize discs or similar material impregnated with various enzyme substrates, carbohydrates, or with various chemical agents for differentiating microorganisms on the identification section of the kit plate. Each of these differentiation discs may be used for presumptive identification of specific organisms. The carbohydrate discs are for the differentiation of microorganisms based on carbohydrate fermentation patterns. In addition, an anaerobe differentiation disc set may be used in the presumptive identification of gram-negative anaerobic bacilli.

Databases can be developed for searching gram-positive microorganisms as is shown for gram-negative microorganisms in the kit and method. It is possible to generate a set of criteria from the kit results for these and other types of microorganisms. In addition, it is practical to generate additional criteria using additional methods of biochemistry for more definitive identification.

The process of preserving the kit plates for later use comprise the packaging and storage under a nitrogen atmosphere performed in a glove box in a low permeability bag. Other embodiments would be to package under nitrogen in a Mylar-foil bag for complete protection against oxygen. Another inert gas could be used to package the kit plates also with another type of impermeable bag or container.

Accordingly, the scope of the invention should be determined not by the embodiments(s) illustrated, but by the appended claims and their legal equivalents.

I claim:

1. A kit for quickly performing a plurality of microbiological test(s) on a broth culture, wherein more than one type of microorganism may exist and comprising:
a. said broth culture, previously inoculated with a microbial sample, providing sufficient numbers of microorganisms for said microbiological test(s) and
b. a kit plate comprising a plurality of test chambers comprising a plurality of identification testing media and antimicrobial susceptibility testing media and
c. antimicrobial impregnated carriers for use with said antimicrobial susceptibility testing media
whereby rapid said microbiological tests, comprising concurrent identification testing and antimicrobial susceptibility testing of one to several microorganism types from said microbial sample, may be performed

2. The kit of claim 1 wherein said broth comprises a rich liquid media sufficient for rapid growth of microorganisms.

3. The kit of claim 1 wherein said broth may be selective for a particular type of microorganism.

4. The kit of claim 1 wherein said broth may support the growth of anaerobic microorganisms.

5. The kit of claim 1 wherein said kit plate comprises a plurality of said test chambers comprising a plurality of solid media

6. The kit of claim 1 wherein said kit plate is polystyrene with a lid of similar composition.

7. The kit of claim 1 wherein said test chambers of said kit plate are rectangular with sides of any convenient dimension

8. The kit of claim 1 wherein said kit plate comprises at least 10 said test chambers comprising selective said identification testing media and said susceptibility testing media.

9. The kit of claim 1 wherein said kit plate comprises at least 10 chambers comprising differential said identification testing media and said susceptibility testing media

10. The kit of claim 1 wherein said kit plate comprises at least 10 chambers comprising differential-selective said identification testing media and said susceptibility testing media

11. The kit of claim 1 wherein said kit plate comprises at least 10 chambers comprising single purpose said identification testing media and said susceptibility testing media

12. The kit of claim 1 wherein said kit plate comprises at least 10 chambers comprising enriched said identification testing media and said susceptibility testing media

13. The kit of claim 1 wherein said kit plate comprises at least 10 chambers comprising a combination of said enriched, said selective, said special purpose, said differential-selective, and said differential identification testing media and said susceptibility testing media.

14. The kit of claim 1 wherein said antimicrobial impregnated carriers a reproduced from standard Kirby-Bauer disk-diffusion antimicrobial disks divided into quarters for placement onto a corner of the susceptibility chamber

15. The kit of claim 1 wherein said antimicrobial impregnated carriers can be constructed from any material that acts as an inert carrier of the antimicrobial agent.

16. A method for quickly performing a plurality of said microbiological test(s) on said broth cultured microbial sample where several different microorganism types may exist and comprising the steps of:
a. providing said broth for rapid cultivation of said microbial sample and
b. providing growth of said microbial sample's microorganisms in said broth culture and providing said kit plate with a plurality of test chambers comprising microbiological testing media and
d. providing inoculation of said kit plate with dilutions from said broth culture and
e. providing incubation of said kit plate for sufficient time to reveal colonies, biochemistries and susceptibilities and
f. providing said microbiological testing on said kit plate comprising said identification testing
and said antimicrobial susceptibility testing which may involve more than one said microorganism type in the same said test chamber and
whereby rapid said microbiological tests comprising concurrent said identification testing and said antimicrobial susceptibility testing of one to several said microorganism types from said microbial sample may be performed.

17. The method of claim 16 wherein said broth is incubated from 4 to 8 hrs from an inoculation of said microbial sample.

18. The method of claim 17 wherein the incubated broth is diluted to several different concentrations and inoculated onto said kit plate where the more dilute inoculation produces individual colonies of said microorganisms for identification.

19. The method of claim 18 wherein said individual colonies are further analyzed to reveal identification of the different types of said microorganisms contained in the same said test chambers.

20. The method of claim 18 wherein the greater concentration dilution produces a lawn of the several said microorganism types within the same said test chambers, and where comprising said susceptibility testing media, these different microorganism types are susceptibility tested.
